(19) United States
(12) Patent Application Publication
(10) Pub. No.: US 2020/0352889 A1
(43) Pub. Date: Nov. 12, 2020

(54) CYSTATHIONINE COMPOSITIONS AND METHODS OF TREATMENT FOR HEALTH CONDITIONS

(71) Applicants: THE REGENTS OF THE UNIVERSITY OF COLORADO, A BODY CORPORATE, Denver, CO (US); MCMASTER UNIVERSITY, Hamilton (CA)

(72) Inventors: Kenneth Maclean, Lakewood, CO (US); Richard Austin, Ancaster (CA)

(21) Appl. No.: 16/885,031

(22) Filed: May 27, 2020

Related U.S. Application Data
(63) Continuation of application No. 15/800,840, filed on Nov. 1, 2017, now abandoned.
Application No. 15/800,840 is a continuation of application No. 14/119,390, filed on Feb. 24, 2014, now abandoned.
(60) Provisional application No. 61/493,035, filed on Jun. 3, 2011.

(51) Int. Cl. A61K 31/198

(52) U.S. Cl. CPC A61K 31/198

(57) ABSTRACT

The present invention relates to uses of and methods of treatment with cystathionine. In one embodiment, cystathionine reduces the development of toxin-induced liver and/or kidney disease induced by homocystinuria and/or acute nephropathy. In one embodiment, a cystathionine synthesis inhibitor is administered to increase tumor cell apoptosis and/or increase the efficacy of chemotherapeutic treatment. More particularly, cystathionine can protect cells against toxin-induced cellular apoptosis and/or cystathionine synthesis inhibitors can increase neuroblastoma cell kill rates during chemotherapy.

### CROSS-REFERENCE TO RELATED APPLICATIONS

The present application is a continuation of, and claims priority to, U.S. patent application Ser. No. 15/800,840, filed Nov. 1, 2017, which is a continuation of U.S. patent application Ser. No. 14/119,390, filed Feb. 24, 2014, which is the U.S. National Phase Application filed under 35 U.S.C. § 371 claiming priority to PCT International Application No. PCT/US2012/040478, filed Jun. 1, 2012, which claims priority under 35 U.S.C. § 119(e) to U.S. Provisional Application No. 61/493,035, filed Jun. 3, 2011, all of which applications are hereby incorporated herein by reference in their entireties.

### FIELD OF THE INVENTION

The present invention relates to uses of and methods of treatment with cystathionine. For example, cystathionine may reduce the development of toxin-induced liver and/or kidney disease induced by homocystinuria and/or acute nephropathy. In one embodiment, a cystathionine synthesis inhibitor may increase the efficacy of chemotherapeutic treatments.

### BACKGROUND OF THE INVENTION

Diseases of the liver and kidney decreases the ability of these organs to function properly. It is estimated that 26 million Americans have kidney disease and millions of others are at increased risk. The kidneys provide the ability to remove wastes and fluids from your body. Both the liver and kidneys play an important role in detoxification such as removal of toxins from the body.

Neuroblastoma is a leading childhood form of cancer that arises in the adrenal gland or in tissue in the nervous system related to the adrenal gland. It is the most common solid tumor outside the brain in infants and children. It is often present at birth but is usually not detected until later on in infancy or childhood. It can affect children up to the age of 10, although diagnosis of older ages has been reported. Treatment of cancer and more particularly of neuroblastoma has included use of monoclonal antibodies that target specific antigens on tumor surfaces such as GD-2-disialoganglioside (Castel et al., Clin Transl Oncol, 12(12):788-93, (2010)). Other treatments have included garlic (Karmakar et al., Anticancer Agents Med Chem. Apr. 26 (2011) e-published). Moreover, many patients with neuroblastoma have elevated levels of cystathionine (Rajnherc et al., Med Pediatr Oncol. 12(2):81-4, (1984); Geiser et al., Cancer October 22(4):856-60, (1968)).

Currently there are no known pharmaceutical therapies for either apoptotic tissue diseases or neuroblastoma. Thus, there is a need to understand the molecular pathogenesis of these diseases as they relate to toxicity and apoptosis in order to develop safe and effective clinical interventions.

### SUMMARY OF THE INVENTION

The present invention relates to uses of and methods of treatment with cystathionine. For example, cystathionine may reduce the development of toxin-induced liver and/or kidney disease induced by homocystinuria and/or acute nephropathy. In one embodiment, a cystathionine synthesis inhibitor may increase the efficacy of chemotherapeutic treatments.

Furthermore, descriptions of embodiments presented are not meant to be limiting and include all equivalent, comparable technologies, reagents, sources, diluents, uses etc. as known by one skilled in the art. In one embodiment, the anti-apoptotic drug is administered with a pharmaceutically acceptable carrier.

In one embodiment the present invention contemplates a method, comprising: a) providing: i) a patient exhibiting at least one symptom of an apoptotic tissue disease; ii) an anti-apoptotic compound, wherein said anti-apoptotic compound is cystathionine, b) administering said anti-apoptotic drug to said patient under conditions such that said at least one symptom of an apoptotic disease is reduced. In yet another embodiment the method includes, wherein said apoptotic tissue disease is in the kidney. In one embodiment, the kidney apoptotic tissue disease comprises endoplasmic reticulum stress-induced cell death. In still other embodiments the methods include, wherein said symptom is selected from the group consisting of nausea, vomiting, fluid retention, increased BUN, increased serum creatinine, and decreased urine output. In some embodiments, the method includes, wherein said administering is selected from the group consisting of parenteral, inhalation, intraperitoneal, intramuscular, subcutaneous, and oral.

In another embodiment, the present invention contemplates a method, comprising: a) providing: i) a patient exhibiting at least one symptom of a neuroblastoma tumor; ii) a cystathionine synthesis inhibitor; b) administering said inhibitor to said patient under conditions such that said at least one symptom of a neuroblastoma tumor is reduced. In one embodiment, the at least one symptom comprises cystathioninuria. In yet another embodiment, the method includes, wherein said administering further comprises that the at least one symptom of cystathioninuria is reduced. In yet other embodiments, the method includes, wherein said administering comprises an amount of the cystathionine synthesis inhibitor capable of chemosensitizing said tumor. In one embodiment, the cystathionine chemosensitizes the tumor. In still further embodiments, the method includes, wherein said symptom is selected from the group consisting of nausea, vomiting, diarrhea, weight loss, rapid pulse, pain, tumor size, and chronic cough. In still other embodiments, the method includes, wherein said administering is selected from the group consisting of parenteral, inhalation, intraperitoneal, intramuscular, subcutaneous, and oral. In one embodiment, inhibitors of cystathionine synthesis are contemplated. For example only, and not meant to be limiting, inhibitors include propargylglycine (PPG).

In one embodiment, the anti-apoptotic drug is optionally administered without a pharmaceutically acceptable carrier. In one embodiment the inhibitor is administered with a pharmaceutically acceptable carrier. In one embodiment the inhibitor is optionally administered without a pharmaceutically acceptable carrier.

In one embodiment cystathionine could be used to block acute kidney damage caused by anti-rejection calcineurin inhibitor drugs such as cyclosporine used after kidney transplantation. Thus, it is believed cystathionine may reduce the development of toxin-induced liver and/or kidney disease induced by acute nephropathy (the data also suggest that this treatment can protect liver and kidney from conditions that are independent of homocystinuria (i.e. ER stress, oxidative stress), as shown in FIGS. 5-8.).

Moreover, in some embodiments cystathionine can protect cells against toxin-induced cellular apoptosis and/or cystathionine synthesis inhibitors can increase neuroblastoma cell kill rates during chemotherapy. In some embodiments, it is believed the anti-apoptotic effects of cystathionine may also protect against tissue damage, steatosis, and necrosis. Further, in some embodiments, it is believed that the use of cystathionine for protecting against liver and/or kidney damage may use a CGL inhibitor, without being limiting, such as propargylglycine to induce accumulation of cystathionine.

### BRIEF DESCRIPTION OF THE FIGURES

FIG. 1 presents one embodiment of methionine metabolism in mammals. For example, a transsulfuration pathway and the methionine and folate cycles are shown.

FIG. 2 presents exemplary data showing that hepatic steatosis induced by a methionine-choline deficient diet is significantly attenuated in HO CBSDH mice with 4-fold elevated cystathionine. (A) H and E staining from representative liver sections of C57BL/6 WT control (upper panel) and HO (lower panel) mice fed an MCD diet for 19 days. Data is representative of n=4-6 per group. Scale bar denotes 200 micrometers. (B) Hepatic triglyceride levels from HO and C57BL/6 WT mice fed an MCD or control diet (n=4). In this and all subsequent figures *, **, and *** denote P values of <0.05., 0.01 and 0.001 respectively.

FIG. 3 presents exemplary data showing that tunicamycin-induced hepatic steatosis and renal tubule damage is significantly attenuated in HO CBSDH mice with 4-fold elevated cystathionine. Representative H and E staining of liver and kidney sections from HO and C57BL/6 WT mice harvested 3 days after treatment with tunicamycin (Tuc). Data is representative of n=4-6 per group. Scale bar denotes 200 micrometers.

FIG. 4 presents exemplary data showing that daily PPG injection induces elevated plasma cystathionine in WT mice. Plasma levels in C57BL/6 WT mice of (A) Cystathionine and (B) Hcy, methionine and cysteine before and one, two, three and four days after treatment with the CGL inactivating compound PPG. Values are means±SD; n=7 at each time point.

FIG. 5 presents exemplary data showing that PPG induced elevated cystathionine significantly protects WT mice against tunicamycin induced steatosis and liver injury. (A) Representative examples of whole livers dissected out of C57BL/6 WT mice from the four experimental groups. Livers from the tunicamycin (Tuc) group were all severely enlarged, yellowish and fatty in appearance. Liver morphology appeared ostensibly normal in mice pretreated with PPG prior to tunicamycin administration. (B) Liver weight as an index of hepatic enlargement (C) Hepatic triglycerides as an index of steatosis and (D) plasma ALT as an index of liver injury were determined. Values are means±SD; n=6.

FIG. 6 presents exemplary data showing that PPG induced elevated cystathionine significantly attenuates steatotic renal enlargement in WT mice treated with tunicamycin. (A) Representative examples of whole kidneys dissected out of C57BL/6 WT mice from the four experimental groups. Kidneys from the tunicamycin (Tuc) group were all severely enlarged and were encased in a relatively large amount of fat. In mice treated with PPG prior to Tuc administration this effect was not apparent (B) Kidney weight as an index of enlargement (C) Kidney triglycerides as an index of steatosis were determined. Values are means±SD; n=6.

FIG. 7 presents exemplary data showing that PPG induced elevated cystathionine significantly attenuates steatotic hepatopathy, renal tubular injury and apoptotic cell death in C57BL/6 WT mice treated with tunicamycin. H and E staining of representative sections from (A) Liver and (B) kidneys from WT mice treated with tunicamycin (Tuc) in the presence and absence of elevated cystathionine induced by PPG. Tuc induced hepatic steatosis and acute tubular necrosis with swelling of proximal tubular epithelial cells. These morphological alterations were significantly attenuated in mice treated with PPG prior to tunicamycin administration. Sections shown are representative of each experimental group (n=6) (C) Assessment of apoptosis by Tunel staining of representative kidney sections of from WT mice treated with tunicamycin (Tuc) in the presence and absence of elevated cystathionine induced by PPG. Apoptotic cells appear dark brown.

FIG. 8 presents exemplary data showing that Tunicamycin-Mediated Induction of the UPR is unaffected by PPG induced elevated cystathionine. Liver and kidney sections were prepared from C57BL/6 WT mice three days after treatment with a single IP injection of either tunicamycin or PBS in the presence or absence of PPG. Representative images are shown from each experimental group immunostained with (A), anti-KDEL and (B), anti-GADD153 antibodies. Scale bar denotes 200 micrometers.

FIG. 9 presents exemplary data showing that cystathionine significantly protects against ER stress induced cell death in 293AD and a23 cells. (A) Relative survival of transsulfuration positive 293AD (upper graph) and transsulfuration deficient a23 Cells (lower graph) treated with tunicamycin (5 or 10 μg/ml) in the presence or absence of either PPG or cystathionine (5 mM). Cell viability was assessed by LDH release assay. Shown are means±SD of a typical experiment performed in triplicate and repeated three times. The ability of cystathionine but not PPG to protect against ER-stress induced cell death in transsulfuration negative a23 cells indicates that cystathionine itself is directly cytoprotective and is not simply serving as a cysteine prodrug (B) photomicrograph of 293AD cells in treated with tunicamycin in the presence and absence of PPG Detached floating cells were clearly visible in the Tuc group and were not evident in the PPG plus tunicamycin group indicating that this compound protects cells against the cytotoxic effects of tunicamycin treatment.

FIG. 10 presents exemplary data showing that cystathionine does not exert its cytoprotective effects by modulating the induction levels of either Grp78 or GADD153. (A) Northern blot determination of Grp78 and GADD153 mRNA levels induced by 5 and 10 μg/ml Tunicamycin in the presence and absence of 5 mM PPG in 293AD cells. Cells treated with 5 mM Hcy were included as a positive control for Grp78 induction. Cell treatment conditions, transfer, probe generation and detection was performed. (B) Western blotting analysis of the tunicamycin mediated induction of the UPR molecular chaperones GRP78 and GRP94 in the presence and absence of PPG (5 mM) and cystathionine (5 mM) in 293AD cells. Cell treatment conditions, transfer, antibody and detection were performed.

FIG. 11 presents exemplary data showing cystathionine can protect against Thapsagargin induced cell death. The figure represents the pooled results of 3 independent experiments each performed in triplicate. It is believed that 1. No cell death was observed in the untreated or cystathionine controls; 2. Cystathionine exerted highly significant protective effects against thapsagargin induced cell death; and 3. The apoptosis inhibitor compound only reduced total cell death by approximately 20% indicating the cellular protective effects of cystathionine are not solely due to the inhibition of apoptosis.

FIG. 12 presents exemplary data showing cystathionine protects against ER stress induced cell death independent of its ability to block apoptosis. The figure represents the pooled results of 3 independent experiments each performed in triplicate. It is believed that 1. Z-VAD-FMK exerted only modest protection against cell death induced by either tunicamycin or thapsagargin (p>0.05 for both) and 2. Cystathionine exerted much greater protective effects against tunicamycin and thapsagargin indicating that much of its protective effects are independent of its role in blocking apoptosis.

FIG. 13 presents exemplary data showing cystathionine protects against acute tubular necrosis induced by the ER stress agent thapsagargin. Wild type (WT) mice (n=6) were exposed to a single sub-lethal intraperitoneal injection of thapsagargin in the presence and absence of the CGL inactivating compound propargylglycine (PPG). WT mice were randomly assigned to one of four treatment groups (n=6 for each group): 1) control, I.P. injected with PBS (See Panel A); 2) I.P. injection with PPG (See Panel B); 3) single LP. tunicamycin, with thapsagargin (See Panel C); and 4) thapsagargin+PPG (See Panel D). In groups 2 and 4, PPG treatment was maintained by daily injection until sacrifice at three days post vehicle/thapsagargin injection. After the completion of this treatment regime, all mice were anaesthetized (isoflourane/oxygen) and sacrificed by decapitation.

### DEFINITIONS

To facilitate the understanding of this invention a number of terms (set off in quotation marks in this Definitions section) are defined below. Terms defined herein (unless otherwise specified) have meanings as commonly understood by a person of ordinary skill in the areas relevant to the present invention.

Unless otherwise indicated, all numbers expressing quantities of ingredients, properties such as molecular weights, reaction conditions, and so forth as used in the specification and claims are to be understood as being modified in all instances by the term “about.” Accordingly, unless indicated to the contrary, the numerical parameters in the specification and claims are approximations that may vary depending upon the desired properties sought to be obtained by the present invention. At the very least, and without limiting the application of the doctrine of equivalents to the scope of the claims, each numerical parameter should at least be construed in light of the number of reported significant digits and by applying ordinary rounding techniques. Notwithstanding that the numerical ranges and parameters describing the broad scope of the invention are approximations, the numerical values in the specific examples are reported as precisely as possible. Any numerical value, however, inherently contains standard deviations that necessarily result from the errors found in the numerical value's testing measurements.

As used herein, a list of abbreviations of terms is as follows:

Alanine aminotransferase (ALT), cystathionine beta-synthase (CBS), CBS deficient homocystinuria (CBSDH), cystathionine gamma-lyase (CGL), endoplasmic reticulum (ER), hematoxylin and eosin (H and E), homocysteine (Hcy), total homocysteine (tHcy), lactate dehydrogenase (LDH), Propargylglycine (PPG), tunicamycin (Tuc), wild type (WT), thapsagargin (Thap), apoptotic inhibitor (Z-VAD-FMK), mouse model of CBSDH designated “HO”. Each of which are known by those skilled in the art and for example can be sourced at but not limited to common commercial/non-commercial suppliers of biological/biotechnology materials such as Sigma-Aldrich, Pierce Chemical, and VWR among others.

As used herein, the term “symptom”, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans. For example only, and not meant to be limiting symptoms include loss of appetite, weight loss, renal/liver injury, loss of condition, enlargement of kidney(s)/liver, cell/tissue death and/or injury, tumor growth, ALT levels, jaundice, lethargy, mental retardation, accumulation of fat around the organs and ER stress among others.

As used herein, the term “disease”, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, toxins or climate); ii) specific infective agents (as worms, bacteria, or viruses); iii) inherent and/or acquired defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

As used herein, the term “administered” or “administering” refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

As used herein, the term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally occurring. A wild-type gene is that which is most frequently observed in a population and is thus arbitrarily designated the “normal” or “wild-type” form of the gene.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “polymerase chain reaction” (“PCR”) refers to the method of K. B. Mullis U.S. Pat. Nos. 4,683,195, 4,683,202, and 4,965,188, hereby incorporated by reference that describes a method for increasing the concentration of a segment of a target sequence in a DNA mixture without cloning or purification. Because the desired amplified segments of the target sequence become the predominant sequences (in terms of concentration) in the mixture, they are said to be “PCR amplified.” Similarly, the term “modified PCR” as used herein refers to amplification methods in which a RNA sequence is amplified from a DNA template in the presence of RNA polymerase or in which a DNA sequence is amplified from an RNA template the presence of reverse transcriptase.

As used herein, the terms “restriction endonucleases” and “restriction enzymes” refer to bacterial enzymes, each of which cut double-stranded DNA at or near a specific nucleotide sequence.

As used herein, the term “antibody” refers to polyclonal and monoclonal antibodies. Polyclonal antibodies which are formed in the animal as the result of an immunological reaction against a protein of interest or a fragment thereof, can then be readily isolated from the blood using well-known methods and purified by column chromatography, for example. Monoclonal antibodies can also be prepared using known methods (See, Winter and Milstein, Nature, 349, 293-299, 1991). As used herein, the term “antibody” encompasses recombinantly prepared, and modified antibodies and antigen binding fragments thereof, such as chimeric antibodies, humanized antibodies, multifunctional antibodies, bispecific or oligo-specific antibodies, single-stranded antibodies and F(ab) or F(ab).sub.2 fragments. The term “reactive” when used in reference to an antibody indicates that the antibody is capable of binding an antigen of interest. For example, a KDEL-reactive antibody is an antibody, which binds to KDELAg or to a fragment of KDELAg.

As used herein, the terms “antigen,” “immunogen,” “antigenic,” “immunogenic,” “antigenically active,” and “immunologically active” refer to any substance that is capable of inducing a specific humoral and/or cell-mediated immune response. An immunogen generally contains at least one epitope. Immunogens are exemplified by, but not restricted to molecules, which contain a peptide, polysaccharide, nucleic acid sequence, and/or lipid. Complexes of peptides with lipids, polysaccharides, or with nucleic acid sequences are also contemplated, including (without limitation) glycopeptide, lipopeptide, glycolipid, etc. These complexes are particularly useful immunogens where smaller molecules with few epitopes do not stimulate a satisfactory immune response by themselves.

As used herein, the term “modulate,” as used herein, refers to a change in the biological activity of a biologically active molecule. Modulation can be an increase or a decrease in activity, a change in binding characteristics, or any other change in the biological, functional, or immunological properties of biologically active molecules.

As used herein the term “portion” when in reference to a protein (as in “a portion of a given protein”) refers to fragments of that protein. The fragments may range in size from four amino acid residues to the entire amino acid sequence minus one amino acid.

As used herein, the term “immunoglobulin” or “antibody” refer to proteins that bind a specific antigen. Immunoglobulins include, but are not limited to, polyclonal, monoclonal, chimeric, and humanized antibodies, Fab fragments, F(ab′).sub.2 fragments, and includes immunoglobulins of the following classes: IgG, IgA, IgM, IgD, IgE, and secreted immunoglobulins (sIg). Immunoglobulins generally comprise two identical heavy chains and two light chains. However, the terms “antibody” and “immunoglobulin” also encompass single chain antibodies and two chain antibodies.

As used herein, the term “antigen binding protein” refers to proteins that bind to a specific antigen. “Antigen binding proteins” include, but are not limited to, immunoglobulins, including polyclonal, monoclonal, chimeric, and humanized antibodies; Fab fragments, F(ab′).sub.2 fragments, and Fab expression libraries; and single chain antibodies.

As used herein, the term “epitope” as used herein refers to that portion of an antigen that makes contact with a particular immunoglobulin.

As used herein, when a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (e.g., the “immunogen” used to elicit the immune response) for binding to an antibody.

As used herein, the terms “specific binding” or “specifically binding” when used in reference to the interaction of an antibody and a protein or peptide means that the interaction is dependent upon the presence of a particular structure (e.g., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabeled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “ligand” denotes a naturally occurring specific binding partner of a receptor, a synthetic specific-binding partner of a receptor, or an appropriate derivative of the natural or synthetic ligands. The determination and isolation of ligands is well known in the art (Lerner, Trends Neurosci. 17:142 146, 1994)). As one of skill in the art will recognize, a molecule (or macromolecular complex) can be both a receptor and a ligand. In general, the binding partner having a smaller molecular weight is referred to as the ligand and the binding partner having a greater molecular weight is referred to as a receptor.

As used herein, the term “in vitro” refers to events that occur in an artificial environment, e.g., in a test tube or reaction vessel, in cell culture, etc., rather than within a multi-cellular organism. For example only and not meant to be limiting HepG2 cells are a human liver carcinoma cell line and are suitable for use as an in vitro model. Further, A293AD (e.g. A293 in literature, see Ren et al., “PPARα Activation Upregulates Nephrin Expression in Human Embryonic Kidney Epithelial Cells and Podocytes,” Biochemical Biophysical Research Communications 305(1):136-142 (2003); Lüdecke et al., “Recessively Inherited L-DOPA-Responsive Parkinsonism in Infancy Caused by A Point Mutation (L205P) in the Tyrosine hydroxylase Gene,” Human Molecular Genetics, 5(7):1023-1028, (1996); cells provided by St. Jude Dept. of Pediatrics, Clinical Genetics and Metabolism section tissue culture collection) by are a human embryonic kidney cell line. As known by those of skill in the art various cell lines are available at but not limited to ATCC among others.

As used herein, the term “in vivo” refers to events that occur within a multi-cellular organism such as a non-human animal.

As used herein, the term “subject” or “patient” refers to any organism to which compositions in accordance with the embodiments of the invention may be administered, e.g., for experimental, diagnostic, prophylactic, and/or therapeutic purposes. Typical subjects include animals (e.g., mammals such as mice, rats, rabbits, non-human primates, dogs, horses, cats, sheep, cattle, pigs, and humans; insects; worms; etc.).

As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

As used herein, the term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

As used herein, the term “test compound”, “compound”, and “agent” refers to any compound or molecule considered a candidate as an inhibitory compound. Furthermore, the term “inhibition”, “inhibitory” and grammatical equivalents thereof includes total, partial, and any gradation thereof.

As used herein, the term “protein” refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

As used herein, the term “peptide” refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

As used herein, the term “pharmaceutically” or “pharmacologically acceptable” refer to molecular entities and compositions that do not produce adverse, allergic, or other untoward reactions when administered to an animal or a human.

As used herein, the term, “pharmaceutically acceptable carrier” includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

As used herein, the term, “purified” or “isolated” may refer to a peptide composition that has been subjected to treatment (i.e., for example, fractionation) to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the composition (i.e., for example, weight/weight and/or weight/volume). The term “purified to homogeneity” is used to include compositions that have been purified to ‘apparent homogeneity” such that there is single protein species (i.e., for example, based upon SDS-PAGE or HPLC analysis). A purified composition is not intended to mean that some trace impurities may remain.

As used herein, the term “substantially purified” refers to molecules, either nucleic or amino acid sequences, that are removed from their natural environment, isolated or separated, and are at least 60% free, preferably 75% free, and more preferably 90% free from other components with which they are naturally associated. An “isolated polynucleotide” is therefore a substantially purified polynucleotide.

The term “Southern blot” refers to the analysis of DNA on agarose or acrylamide gels to fractionate the DNA according to size, followed by transfer and immobilization of the DNA from the gel to a solid support, such as nitrocellulose or a nylon membrane. The immobilized DNA is then probed with a labeled oligodeoxyribonucleotide probe or DNA probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists. J. Sambrook et al. (1989) In: Molecular Cloning: A Laboratory Manual, Cold Spring Harbor Press, NY, pp 9.31-9.58.

The term “Northern blot” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled oligodeoxyribonucleotide probe or DNA probe to detect RNA species complementary to the probe used. Northern blots are a standard tool of molecular biologists. J. Sambrook, J. et al. (1989) supra, pp 7.39-7.52.

As used herein, the term “apoptosis” is the process of programmed cell death (PCD) that might occur in multi-cellular organisms. Biochemical events lead to characteristic cell changes (morphology) and death. For example, changes include but are not limited to blebbing, cell shrinkage, nuclear fragmentation, chromatin condensation, and chromosomal DNA fragmentation. For example, known apoptosis inhibitors include but are not limited to Z-VAD-FMK (a caspase inhibitor available from Promega Corporation, Sigma-Aldrich among others). This contrasts with “necrosis”, which is a form of traumatic cell death that results from acute cellular injury. Necrosis is caused by factors external to the cell or tissue, such as infection, toxins, or trauma.

### DETAILED DESCRIPTION OF THE INVENTION

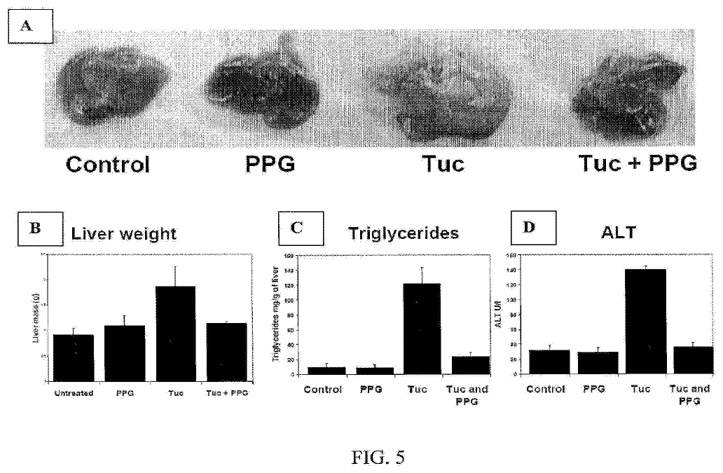

The present invention relates to uses of and methods of treatment with cystathionine. For example, cystathionine may reduce the development of toxin-induced liver and/or kidney disease induced by homocystinuria and/or acute nephropathy (the data also suggest that this treatment can protect liver and kidney from conditions that are independent of homocystinuria (i.e. ER stress, oxidative stress), as shown in FIGS. 5-8.) In one embodiment, a cystathionine synthesis inhibitor may increase the efficacy of chemotherapeutic treatments.

Some prior publications disclosing use of cystathionine include: Ghibelli et al., “Rescue of Cells from Apoptosis by Inhibition of Active GSH Extrusion,” FASEBJ, 12:479-486, (1998) discussing use of cystathionine as an inhibitor of apoptosis in cells undergoing glutathione (GSH) efflux; Kang et al, “Protection of CSE/H2S System in Hepatic Ischemia Reperfusion Injury in Rats,” Zhonghua Wai Ke Za Zhi, 48(12):924-8, (2010) discussing a cystathionine gamma-lyase (CSE)/hydrogen sulfide (H2S) system in rats, might be involved in protection of the liver from hepatic ischemia-reperfusion (IR) injury; Fotakis et al., “Modulation of Cadmium Chloride Toxicity by Sulphur Amino Acids in Hepatoma Cells,” Toxicol In Vitro, 20(5):641-8, (2006) discussing that cystathionine protects HTC cells against cadmium chloride induced apoptosis toxicity but not HepG2 cells; Klein et al., “Cystathionine Metabolism in Neuroblastoma,” Cancer, 62:291-8, (1987) discussing cystathionine as a urinary biomarker for neuroblastoma; and Yokouchi et al., “Atypical, Bidirectional Regulation of Cadmium-Induced Apoptosis Via Distinct Signaling of Unfolded Protein Response,” Cell Death and Differentiation, 14: 1467-1474, (2007) discussing that cadmium can induce apoptosis of tubular epithelial cells. Each of which is herein incorporated by reference.

### Cystathionine

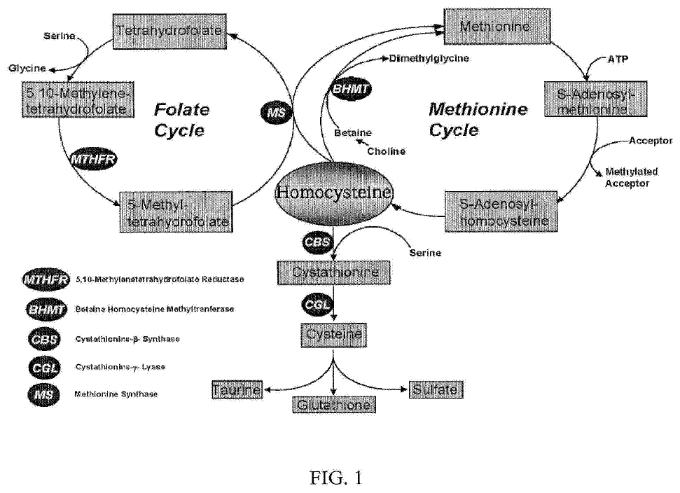

Cystathionine (R—S-(2-Amino-2-carboxyethyl)-L-homocysteine) may be a non-proteinogenic thioether containing amino acid that is not present in significant amounts in the human diet. Endogenous synthesis of cystathionine may be catalyzed by cystathionine β-synthase (CBS) via a pyridoxal 5′-phosphate (PLP) dependent β-replacement reaction condensing serine and homocysteine (Hcy) to form cystathionine, which is subsequently converted to cysteine plus ammonia and α-ketobutyrate by the action of cystathionine γ-lyase (CGL) (FIG. 1) (1).

Pathogenic mutations in CBS result in CBS deficient homocystinuria (CBSDH), which, if untreated, results in mental retardation, thromboembolic complications and a range of connective tissue disorders. Although it is not necessary to understand the mechanism of action, it is believed that most pathogenic mechanisms that underlie CBSDH are poorly understood but the majority of research to date has focused upon the role of elevated homocysteine (Hcy). In addition to causing elevated tissue and plasma, Hcy, methionine, S-adenosylmethionine (AdoMet), and S-adenosylhomocysteine (AdoHcy), CBS inactivation also completely abolishes synthesis of cystathionine. Currently there is no known function for cystathionine other than serving as an intermediate in transsulfuration and to date the possible contribution of the abolition of its synthesis to pathogenesis in CBSDH has not been investigated.

The data presented herein, suggests that cystathionine may be capable of blocking the induction of steatotic liver injury, acute tubular necrosis and/or apoptotic cell death by the endoplasmic reticulum stress inducing agent tunicamycin. For example, Immunohistochemical, Northern and Western blotting analysis indicate that the protective effects of cystathionine occur without significant alteration of the unfolded protein response. While it is not necessary to understand the mechanism of action, one embodiment contemplates that this data may provide the first experimental evidence that the abolition of cystathionine synthesis may contribute to the pathology of CBSDH and it is believed that this compound has therapeutic potential for disease states where ER stress is implicated as a primary initiating pathogenic factor.

Cystathionine May Reduce the Development of Toxin-Induced Liver and/or Kidney Disease Induced by Homocystinuria.

Acute tubular necrosis in the kidney can be caused by many toxic insults including, but not limited to, depleted uranium and drugs commonly used during organ transplantation. While it is not necessary to understand the mechanism of action, it is believed that one embodiment of the current invention contemplates the induction of severe acute tubular necrosis caused by the inhibitor of protein glycosylation, tunicamycin, is significantly ameliorated by the transsulfuration intermediate cystathionine and as such this compound constitutes a novel treatment for this pathogenic condition.

A previously described cbs null mouse model of CBSDH exhibits a semi-lethal phenotype due to severe steatosis, fibrosis and neonatal liver failure (2,3). It is believed that a mouse model of CBSDH designated HO has been generated, that exhibit essentially identical levels of methionine cycle metabolites as the previously described cbs null mouse model but only presents with mild hepatopathy without any detectable hepatic steatosis, fibrosis or neonatal liver failure. The only metabolite to differ significantly from the cbs null model was cystathionine, which due to Hcy mediated inactivation of CGL, accumulated to a level four-fold higher than that observed in normal control animals. Previous work has suggested that hepatopathy in the cbs null model is due to the induction of endoplasmic reticulum (ER) stress by Hcy (4). This possibility is consistent with the fact that electron microscopy analysis found markedly distended ER with fine intracisternal proteinaceous precipitates in liver samples of the cbs null but not in the HO mice (3,5). While it is not necessary to understand the mechanism of action, it is believed that these observations suggest that cystathionine may be able to exert protective effects against hepatic lipid accumulation and tissue injury induced by ER stress. While it is not necessary to understand the mechanism of action, it is believed that HO CBSDH mice exhibiting elevated cystathionine show significantly attenuated hepatic steatosis in the presence of an adipogenic methionine-choline deficient diet.

Additionally, one embodiment of the present invention contemplates that elevated cystathionine, independent of its role as a cysteine donor, significantly attenuates hepatic and renal lipid accumulation, tissue injury and cell death induced by ER stress. Subsequent analysis revealed that cystathionine does not protect against the pathological effects of ER stress by modulating expression levels of the unfolded protein response. Thus, while it is not necessary to understand the mechanism of action, one embodiment of the present invention contemplates that collectively, these findings constitute the first experimental evidence that the abolition of cystathionine synthesis may contribute to the pathology of CBSDH and that it is believed that this compound has therapeutic potential for disease states where ER stress is implicated as a primary initiating pathogenic factor.

### Acute Tubular Necrosis

Acute tubular necrosis is a kidney disorder involving damage to the tubule cells of the kidneys, resulting in acute kidney failure.

### Causes

Acute tubular necrosis (ATN) is caused by lack of oxygen to the kidney tissues (ischemia of the kidneys). The internal structures of the kidney, particularly the tissues of the kidney tubule, become damaged or destroyed. ATN is one of the most common structural changes that can lead to acute renal failure. ATN is one of the most common causes of kidney failure in hospitalized patients. a. Risks for acute tubular necrosis include, but are not limited to: Blood transfusion reaction, Injury or trauma that damages the muscles, Recent major surgery, Septic shock or other forms of shock, Severe low blood pressure (hypotension) that lasts longer than 30 minutes, Liver disease and kidney damage caused by diabetes (diabetic nephropathy) may make a person more susceptible to the condition. b. ATN can be caused by: Exposure to medications that are toxic to the kidneys (such as aminoglycoside antibiotics), Antifungal agents (such as amphotericin), Dye used for x-ray (radiographic) studies.

### Symptoms

Decreased consciousness, Coma, Delirium or confusion, Drowsy, lethargic, hard to arouse, Decreased urine output or no urine output, General swelling, fluid retention, Nausea, vomiting, Other symptoms of acute kidney failure may also be present.

### Exams and Tests

Examination usually indicates acute kidney failure. There may be signs of fluid overload, including abnormal sounds on listening to the heart and lungs with a stethoscope (auscultation).

### Other Signs Include, but are not Limited to:

BUN and serum creatinine levels may increase Fractional excretion of sodium and of urea may be relatively high Kidney biopsy may show acute tubular necrosis (but a biopsy is rarely done), Urinalysis may show casts, kidney tubular cells, and red blood cells, Urine sodium may be high, and Urine specific gravity and osmolarity urine indicate dilute urine.

### Characterization of Cystathionine Treatment

### HO CBSDH Mice Exhibit Significantly Attenuated Progression of Diet-Induced Hepatic Steatosis

To date, all cbs null mouse models of CBSDH have been found to incur liver injury and a high degree of neonatal lethality (2,3,15). Those cbs null mice that survive the neonatal period invariably incur profound hepatic steatosis (2,3). Despite having essentially identical levels of plasma and tissue Hcy as the cbs null models, the HO CBSDH mice do not exhibit any discernible hepatic steatosis (5) leading us to speculate that the elevated cystathionine exhibited by HO mice might be exerting protective effects against aberrant hepatic lipid accumulation.

To investigate this possibility, the data presented herein evaluates the relative degree of hepatic lipid accumulation in HO and wild type control mice fed a methionine and choline deficient (MCD) diet. Although it is not necessary to understand the mechanism of action, it is believed that this diet rapidly induces hepatic steatosis as methionine and choline are lipotropic precursors of phosphatidylcholine synthesis and their exclusion from the diet inhibits the assembly of very low density lipoprotein (VLDL) causing impairment of the secretion of triglycerides and free fatty acids from hepatocytes (16).

One group of HO mice and one group of WT control mice (n=5 for each) were put on an MCD. Control groups of both genotypes (n=5) were put on a chow diet that matched the composition of the MCD diet except that it contained normal levels of choline and methionine. All groups were kept on their respective diets for a total of 17 days. At the end of the trial, the mice were sacrificed and the livers of these animals were examined. A party unaware of the animals' genotype or treatment group performed histological analysis. No hepatic steatosis was observed in either of the control groups (data not shown).

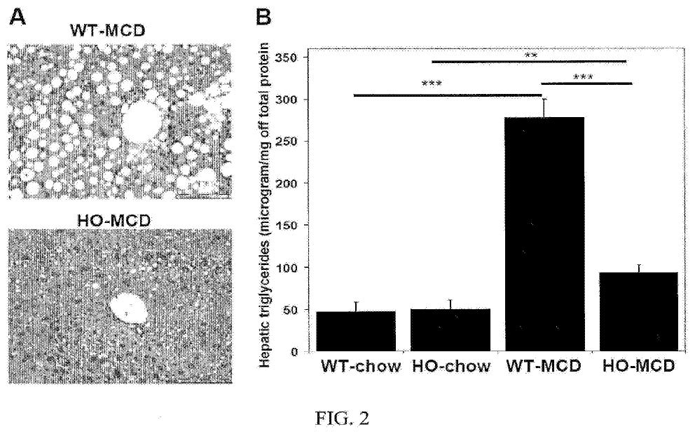

Histological examination of the livers after H and E staining found that the MCD diet induced severe macrovesicular hepatic steatosis, ballooning degeneration of hepatocytes and multiple inflammatory foci in the WT mice. These sequelae were greatly attenuated in the HO CBSDH mice (FIG. 2, Panel A). To quantitate the development of hepatic steatosis induced by the MCD diet, the triglyceride content was measured in the livers of the MCD fed HO and WT mice and in the animals fed the control diet (FIG. 2, Panel B). The lipid levels of both the HO and WT animals on the control chow diet did not differ significantly from each other (p=0.707). Compared to the relevant genotype chow controls, the MCD diet induced an approximate 6-fold increase in the hepatic triglyceride levels of WT animals but only a 2-fold increase in HO mice. This observation is consistent with our histological analysis and collectively, these data support the possibility that elevated cystathionine can exert hepatoprotective effects.

HO Mice Exhibit Ablated Hepatic and Renal Injury after Tunicamycin Treatment

The ER is believed to be the primary organelle for the synthesis, folding and modification of proteins in the eukaryotic cell and exposure of cells to conditions such as inhibition of protein glycosylation, nutrient/oxygen deprivation or perturbation of Ca2+ homeostasis, results in the accumulation of unfolded proteins causing ER stress (17). Previous work has indicated that the MCD diet induces hepatic steatosis at least in part by inducing ER stress (7) Similarly, previous work has suggested that the steatosis observed in cbs null mice is directly related to the action of elevated Hcy inducing ER stress causing an alteration of serum response element binding protein 1 (SREBP1) function that subsequently results in dysfunctional lipid metabolism (4). The ability of the HO mice to resist hepatic steatosis in the presence of an MCD diet and our previous observation of distended ER in the steatotic cbs null mice but not the HO mice raises the possibility that elevated cystathionine in HO mice is exerting its hepatoprotective effects by blocking the induction of ER stress.

To examine this possibility, a tunicamycin-mediated model of ER stress induced tissue injury on groups of HO and WT mice was performed. The nucleoside antibiotic, tunicamycin, produced by the actinomycete, Streptomyces lysosuperifcus, causes ER stress by inhibiting UDP-N-acetylgluco samine:dolichol phosphate N-acetylglucosamine-1-P transferase blocking protein N-glycosylation. Previous work has shown that a single sub-lethal intraperitoneal injection with 0.5 mg/kg body weight of tunicamycin results in a reproducible pattern of lassitude, lack of grooming, weight loss, renal injury and hepatic steatosis that peaks between day 4 and 5 post-injection (9,10). Groups of WT and HO (n=5 for all groups) mice were given either a single intra-peritoneal injection of tunicamycin or sham injected with an identical volume of the vehicle solution PBS. All mice treated with tunicamycin became markedly inappetent shortly after injection and marked weight loss was clearly noticeable and progressive during the course of the experiment. Three days after the administration of treatment, mice were sacrificed and tissues were extracted and processed for histological analysis. In the sham injected control animals no evidence of tissue injury or steatosis was observed (data not shown). In the WT control mice as expected, significant weight loss, lassitude and altered fur was observed.

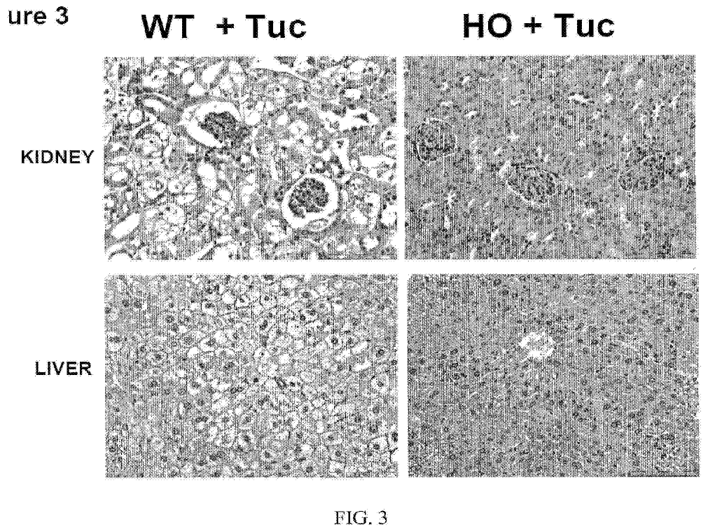

Additionally, it was observed that the livers of the tunicamycin treated wild type animals were severely enlarged and yellowish in color. Histologically, H and E staining revealed a clearly discernible pattern of periportal hepatocellular damage, severe vacuolation of hepatocytes was observed much of which appeared to result from lipid accumulation. Single, condensed, rounded or ovoid cytoplasmic bodies, apparently derived from degenerating hepatocytes, were scattered throughout the liver, but especially prevalent in the periportal region (FIG. 3). The most severe injury was in the kidney where significant enlargement and accumulation of fat around the organ was observed. Histological analysis revealed severe vacuolation, steatosis and acute tubular necrosis with swelling of proximal tubular epithelial cells with pyknotic nuclei and tubules with focal areas of denuded basal lamina. The histological alterations were primarily located in the proximal tubular epithelium. (FIG. 3). These morphological changes are very similar to those described in mice and other species following tunicamycin injection (9,18,19).

HO mice treated with tunicamycin incurred essentially identical weight loss, lassitude and altered grooming as the WT mice but strikingly, did not exhibit any significant liver enlargement with only minimal hepatic steatosis and no discernible alteration of renal morphology (FIG. 3). While it is not necessary to understand the mechanism of action, it is believed that these data are consistent with the possibility that elevated cystathionine acts to protect against ER stress mediated tissue injury in both the liver and kidney.

Daily Injection with the CGL Inactivating Compound Propargylglycine Cause Temporary Hypercystathionemia in Normal Mice

Although the data described above indirectly supports the possibility that cystathionine exerts tissue protective effects at least in part by blocking the pathogenic consequences of prolonged ER stress, there are multiple biochemical differences between the HO mice and WT mice (3,5). In order to provide more direct evidence of a protective role for cystathionine, it was proposed to induce elevated cystathionine in normal mice in order to examine its protective properties in isolation from the elevated Hcy, methionine, AdoMet, AdoHcy and decreased cysteine that is observed in the HO mice.

Inducing elevated cystathionine in normal mice may be complicated by the fact that this compound has relatively low solubility in water and is rapidly excreted in the urine of mice. Previous work by Abeles and Walsh synthesized and tested a range of acetylenic substrates capable of irreversible inactivation of CGL. For example, I.P. injection of one of these inactivators D,L-2-amino-4-pentynoic acid also known as propargylglycine (PPG) has been shown to rapidly inactivate hepatic CGL activity and results in a concomitant accumulation of cystathionine within one hour that lasts for approximately one day (20). For these purposes, some embodiments of the present invention have the additional benefit of allowing investigation of the possible protective effects of elevated cystathionine in isolation from its role as a cysteine donor.

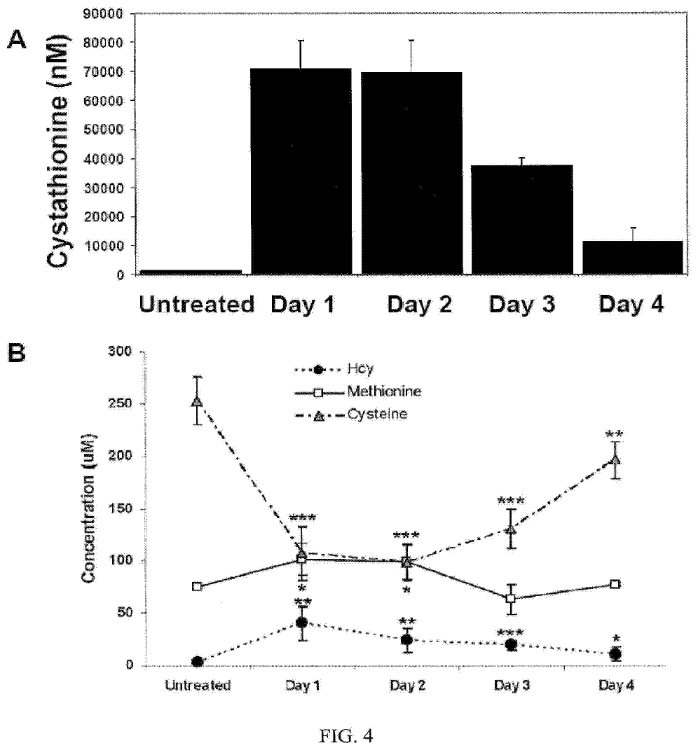

It is believed that the longevity of this cystathionine accumulation with repeated injections of PPG over time in mice has not been properly examined. Consequently, daily injections of PPG were given to WT mice (n=7) each morning over a period of 4 days. Plasma samples were taken from these mice in the evening of each day by non-lethal tail bleed and the levels of cystathionine, Hcy, methionine and cysteine were determined (FIG. 4). Inactivation of CGL was observed and was accompanied by a 56-fold increase in the plasma level of cystathionine on day 1. This maximal accumulation persisted for the second day and was reduced to 30-fold by day 3. By day 4 of the experiment, the level of cystathionine accumulation was reduced to 8-fold compared to untreated animals indicating that despite daily re-administration of this compound, the effect of PPG diminishes over time. Blocking transsulfuration also induced a relatively mild elevation in total Hcy (tHcy) and methionine that declined back to normal as the efficacy of the PPG treatment diminished. As expected, blocking transsulfuration via PPG treatment resulted in a concomitant decrease in plasma cysteine levels during the first and second days of PPG treatment. Cysteine levels subsequently returned to near normal levels as the effects of the PPG treatment diminished.

While it is not necessary to understand the mechanism of action, the above data suggest an optimal time frame for examining the possible protective effects of PPG mediated cystathionine accumulation in WT mice. The effect of PPG on liver health was assessed by determining ALT values before PPG treatment and at each subsequent time point studied. PPG treatment did not induce any significant increase in plasma ALT levels at any of the time points analyzed indicating that this treatment is relatively benign over the time period studied (data not shown).

### Elevated Cystathionine in WT Mice Protects Against Hepatic and Renal Damage Mediated by the ER Stress Inducing Agent Tunicamycin

In order to investigate the effects of elevated cystathionine upon the response to ER stress, wild type mice (n=6) were exposed to a single sub-lethal intraperitoneal injection of tunicamycin in the presence and absence of the CGL inactivating compound PPG. WT mice were randomly assigned to one of four treatment groups (n=6 for each group): 1) control, IP injected with PBS; 2) I.P. injection with PPG; 3) single I.P tunicamycin, with tunicamycin and 4) tunicamycin+PPG. In groups 2 and 4, PPG treatment was maintained by daily injection until sacrifice at three days post vehicle/tunicamycin injection. After the completion of this treatment regime, all mice were anaesthetized (isoflourane/oxygen) and sacrificed by decapitation, and blood samples were collected for plasma analysis. Livers and kidneys were removed, weighed and then samples were either immersion-fixed overnight in 4% paraformaldehyde in PBS (pH 7.3) for subsequent histological analysis or were snap frozen in liquid nitrogen and examined for morphological, histological and biochemical analyses.

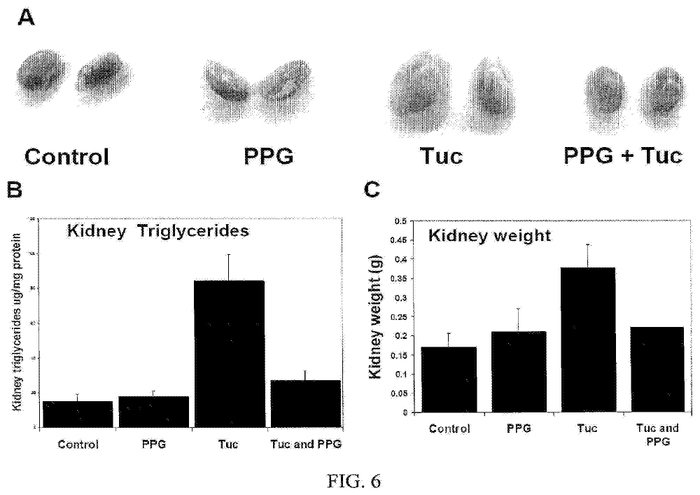

The livers and kidneys of the sham injected and PPG alone control groups were normal in size and appearance while the livers from the animals treated solely with tunicamycin were enlarged, yellowish and fatty in appearance (FIG. 5, Panel A) Similarly, the kidneys of these animals were also swollen and covered in fat (FIG. 6, Panel A). This enlargement was reflected in increased liver and kidney weight compared to the controls (FIG. 5, Panel B, and FIG. 6, Panel B). Determination of triglycerides showed a highly significant increase in both liver (P<0.001) and kidney (p<0.001) of the animals treated solely with tunicamycin compared to the sham and PPG control groups confirming the induction of hepatic and renal steatosis by this toxin (FIG. 5, Panel C, and FIG. 6, Panel C). The induction of significant liver injury by tunicamycin treatment was also confirmed by determination of plasma ALT levels. (FIG. 5, Panel D). While it is not necessary to understand the mechanism of action, it was surprisingly observed that livers and kidneys from the hypercystathionemic mice that were treated with tunicamycin appeared ostensibly normal and did not differ significantly from the control groups in either liver weight, triglyceride levels or plasma ALT levels.

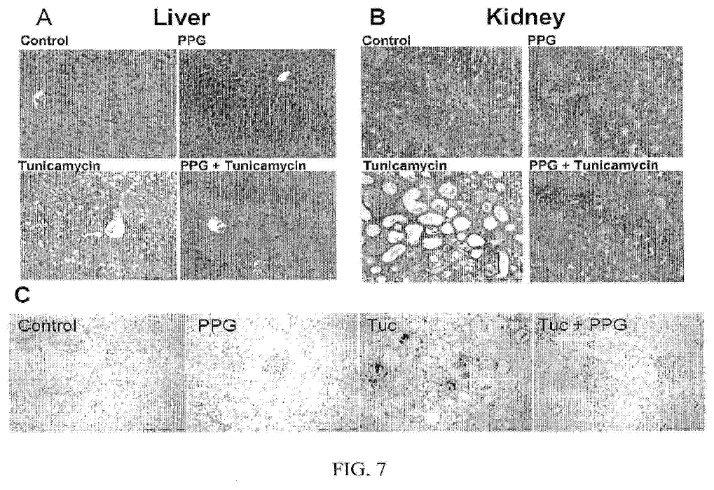

For example, histological analysis revealed that the sham injection and PPG control groups showed no significant morphological alteration while the tunicamycin alone group incurred severe morphological changes in the liver and kidney that were essentially identical to those described above. These morphological changes were effectively ablated in the mice that were induced into hypercystathionemia by PPG treatment during the tunicamycin treatment (FIG. 7, Panels A-B). While it is not necessary to understand the mechanism of action, to further assess the protective effects of cystathionine, the tissues of all of these mice were analyzed for evidence of apoptosis using Tunel staining. No apoptosis was observed in either of the control groups. In the tunicamycin treated animals the most affected organ was the kidney. In this tissue, the proximal tubule epithelium in particular showed clear evidence of apoptotic cell death (FIG. 7, Panel C). No apoptotic cells were observed in the kidneys of tunicamycin treated mice that had been pretreated with PPG. While it is not necessary to understand the mechanism of action, it is believed that collectively, the above data suggests that cystathionine may exert significant protective effects against ER stress mediated lipid accumulation, tissue injury and apoptotic cell death.

PPG Induced Hypercystathionemia does not Alter Induction of the Unfolded Protein Response in Mice

Eukaryotic cells, respond to ER stress by activating a set of pathways known as the unfolded protein response (UPR). The UPR is transmitted through the activation of ER resident proteins, such as protein kinase-like ER kinase (PERK), activating transcription factor 6 (ATF6) and inositol-required enzyme 1 (Irel/β). These signaling pathways induce the expression of a number of molecular chaperones such as the KDEL amino acid signature containing proteins glucose regulated protein (GRP) 78 (also Known as Bip) and 70 that play a role in assisting the correct folding of proteins in an effort to try and ameliorate the accumulation of misfolded proteins and thus alleviate ER stress (21). However, if ER stress is prolonged and the burden of misfolded proteins in the ER exceeds its folding capacity, cells are typically induced into apoptosis. Prolonged perturbation of the ER is a powerful inducer of the C/EBP family member GADD153 (also known as CHOP) that plays a key role in the induction of programmed cell death by ER stress (10). Previous work has shown that GRP78 over-expression in Chinese hamster ovary cells attenuates both the ER stress-signal and the cell death that is observed in response to calcium ionophores (22) and blocking the expression of GRP78 by means of antisense constructs increase the lethality of agents that promote ER stress (23).

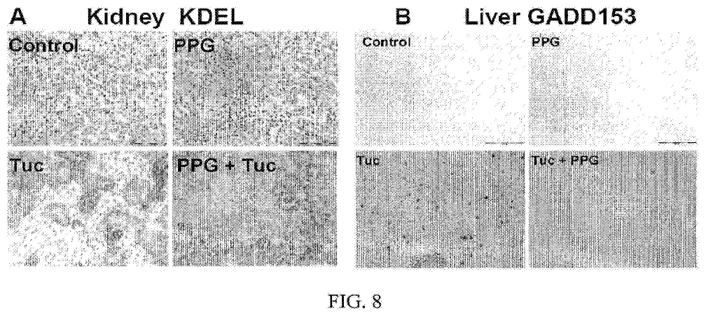

While it is not necessary to understand the mechanism of action, it is believed cystathionine may protect against ER stress induced cell injury by increasing the induction of molecular chaperones like Grp78 or by inhibiting the induction of GADD153. In order to investigate the mechanism by which cystathionine is protecting against ER stress induced tissue injury, an immunohistochemical analysis of liver and kidney tissues samples from the experimental groups described above was performed. PPG treatment alone did not induce expression of any KDEL signature proteins (FIG. 8, Panel A). Strong induction of KDEL containing proteins and GADD153 by tunicamycin treatment in both liver and kidneys of the tunicamycin groups was observed (FIG. 8, Panel B). While it is not necessary to understand the mechanism of action, it is believed that despite the obvious protective effects of PPG-induced hypercystathionemia, this treatment did not appear to affect the level of induction of either the KDEL containing chaperones or GADD153 indicating that cystathionine is not exerting protective effects against ER stress by modulating the UPR or by blocking the induction of GADD153.

Cells Treated with Cystathionine have Increased Resistance to the Death-Promoting Effects of ER Stress

While it is not necessary to understand the mechanism of action, it is believed that the above in vivo model data suggests that in addition to protecting against ER stress induced lipid accumulation and tissue injury, cystathionine may also play a role in promoting cell survival by attenuating the induction of apoptosis. However, examination of a direct effect of exogenously added cystathionine is not currently possible in a whole animal model because of complications with the compounds solubility and rapid kinetics of its excretion. In order to investigate the direct effects of cystathionine against ER stress-induced cell death, the cytotoxicity of tunicamycin in the human embryonic kidney cell line 293AD and the human hepatoma cell line HepG2 in the presence and absence of both PPG and exogenously added cystathionine was examined. Preliminary experiments confirmed that both of these cell lines express both CBS and CGL (data not shown). In order to investigate the possible protective effects of cystathionine in the absence of transsulfuration, these experiments were repeated with the CBS and CGL negative Chinese hamster fibroblast line a23 (6).

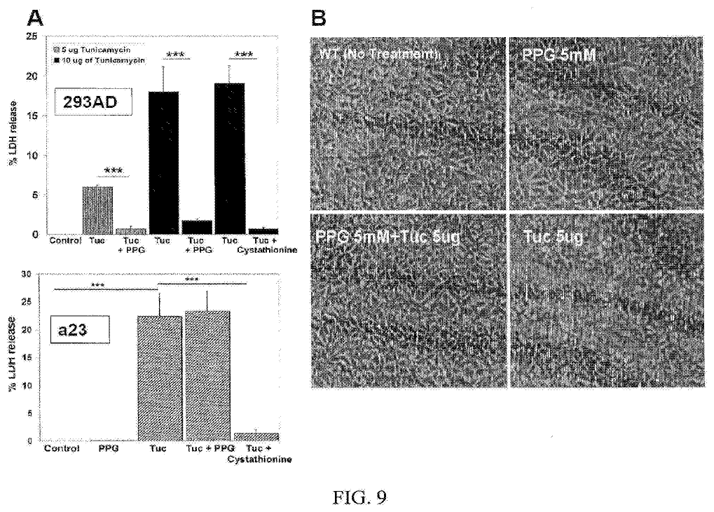

Cells were seeded in triplicate at ˜75% confluence and pre-treated with either PPG) (5 mM), Cystathionine (1 mM) or with just complete media (DMEM, 10% FBS) as a control. After 24 hr pre-treatment, cells looked normal, with minimal floating cells. Fresh media with 1% FBS was then added to each well, the cells were further treated with Tm (either 5 or 10 ug)/ml. After 18 hr. treatment with Tunicamycin, cells were photographed under phase-contrast with a Zeiss Axiovert 25 microscope for evidence of dead floating cells. Cells from the group treated solely with tunicamycin became strikingly hyper-refringent, detached from the substratum and floated in the media. Conversely, No floating dead cells were observed in either the PPG or cystathionine pre-treated cells (FIG. 9, Panel B). To further evaluate a role for cystathionine in resisting the induction of cell death by tunicamycin, cell lysis was quantified by assaying lactate dehydrogenase (LDH) release. The enzyme activity was determined spectrophotometrically and the amount of enzyme leak was expressed as a percentage of the LDH activity observed in untreated control cells.

For example, treatment of 293AD cells with 5 μg/ml of tunicamycin resulted in significant cell death that was largely prevented by pre-treatment with PPG (FIG. 9, Panel A). Treatment of the cells with a higher dose of tunicamycin (10 μg/ml) resulted in an even more pronounced protective effect with PPG and cystathionine causing an 11 and 27-fold reduction in cell death respectively (p<0.0001). Essentially identical results were observed when this experiment was repeated using HepG2 cells (data not shown). When this experiment was repeated with the transsulfuration negative a23 cells, cystathionine was still clearly protective with a 16-fold reduction in tunicamycin induced cell death (p<0.0001) but PPG conferred no significant cytoprotection (p=0.7301). While it is not necessary to understand the mechanism of action, it is believed that this latter observation may discount the possibility that PPG itself or a metabolite thereof is responsible for the protection independent of its role in inducing elevated cystathionine. While it is not necessary to understand the mechanism of action, it is believed that the above data suggests that cystathionine promotes increased survival of cells exposed to toxic levels of ER stress.

Cystathionine Exerts its Protective Effects Against ER Stress Induced Cell Death with Out Modulating Expression of the UPR

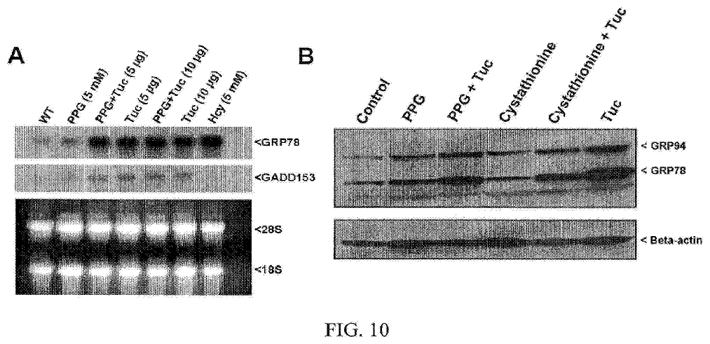

To further assess the effect of cystathionine upon induction of the UPR and GADD153 by ER stress Northern and Western blotting experiments were performed. Neither PPG nor exogenously added cystathionine induced expression of the KDEL containing proteins Grp78 or Grp94 or the cell death-promoting gene GADD15 in the control cells (FIG. 10, Panels A-B). Nor did either of these treatments significantly alter the scale of induction of these proteins in the presence of tunicamycin treatment. While it is not necessary to understand the mechanism of action, it is believed that the above data suggests that these experiments are consistent with the findings in the wild type mouse model described above and further indicate that cystathionine does not exert its cytoprotective effects against ER stress induced cell death by modulating the expression of the UPR.

### Cystathionine can Protect Against Thapsagargin Induced Cell Death

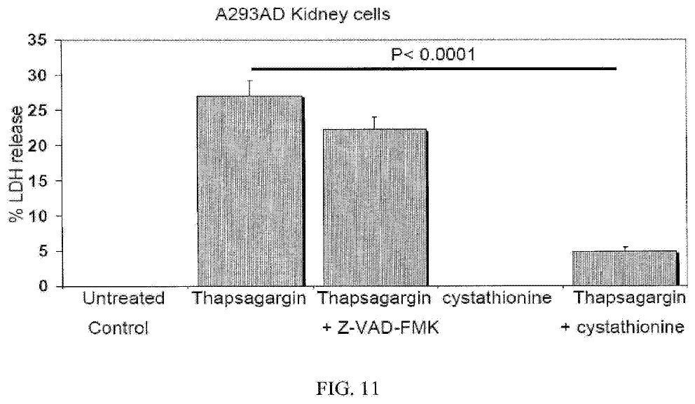

It was previously shown that cystathionine can protect against ER stress induced cell death induced by the glycosylation inhibitor tunicamycin. In order to investigate if this protective effect was limited to tunicamycin, it was investigated whether cystathionine (1 mM) could protect A293AD kidney cells against ER stress induced cell death mediated by the calcium metabolism inhibitor compound thapsagargin (1 uM). Cell death was monitored by measuring LDH release into culture media. In order to investigate if cystathionine was simply blocking apoptosis, the effect of thapsagargin in the presence of the known apoptosis inhibitor Z-VAD-FMK was investigated. FIG. 11 shows the pooled results of 3 independent experiments each performed in triplicate. While it is not necessary to understand the mechanism of action, it is believed that 1. No cell death was observed in the untreated or cystathionine controls; 2. Cystathionine exerted highly significant protective effects against thapsagargin induced cell death; and 3. The apoptosis inhibitor compound only reduced total cell death by approximately 20% indicating the cellular protective effects of cystathionine are not solely due to the inhibition of apoptosis.

### Cystathionine Protects Against ER Stress Induced Cell Death Independent of its Ability to Block Apoptosis

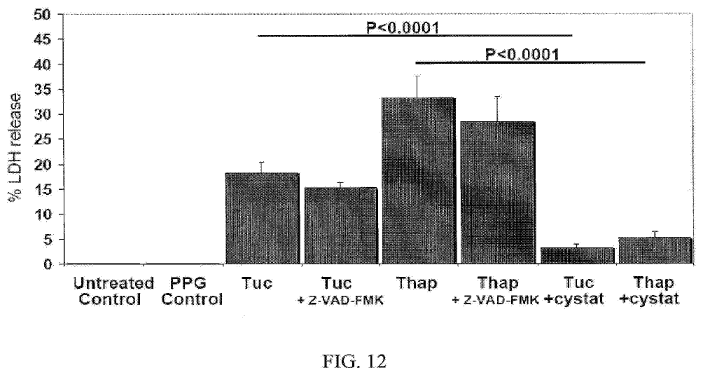

Previous work showed that cystathionine can block ER stress induced apoptosis. The acute renal necrosis seen in mice treated with tunicamycin indicates that much of the tissue damage induced by this compound is necrotic and independent of apoptosis. In order to investigate if cystathionine protects against tunicamycin (tuc) or thapsagargin (Thap) induced cell death solely as an anti-apoptotic compound the ability of the known apoptotic inhibitor compound Z-VAD-FMK to prevent cell death induced by these treatments in HepG2 cells was investigated. FIG. 12 shows pooled results of 3 independent experiments each performed in triplicate. While it is not necessary to understand the mechanism of action, it is believed that 1. Z-VAD-FMK exerted only modest protection against cell death induced by either tunicamycin or thapsagargin (p>0.05 for both) and 2. Cystathionine exerted much greater protective effects against tunicamycin and thapsagargin indicating that much of its protective effects are independent of its role in blocking apoptosis.

### Cystathionine Protects Against Acute Tubular Necrosis Induced by the ER Stress Agent Thapsagargin

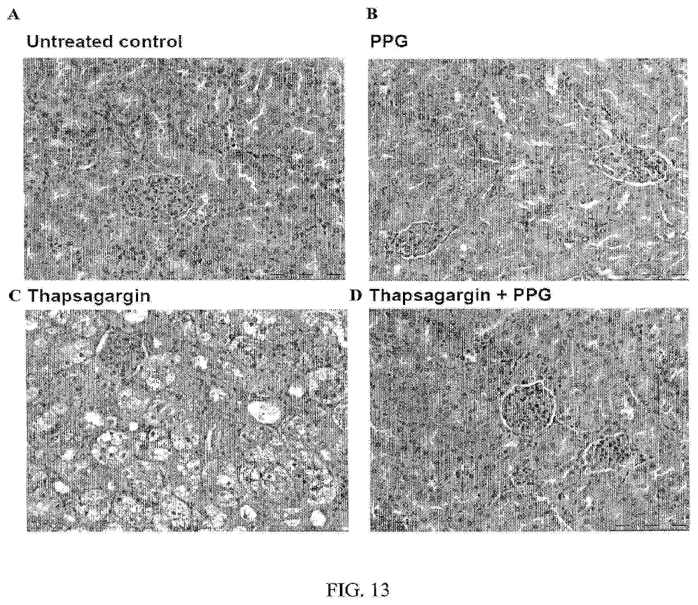

Wild type mice (WT; n=6) to a single sub-lethal intraperitoneal injection of thapsagargin in the presence and absence of the CGL inactivating compound propargylglycine (PPG). WT mice were randomly assigned to one of four treatment groups (n=6 for each group): 1). control, IP injected with PBS (See FIG. 13, Panel A); 2). I.P. injection with PPG (See FIG. 13, Panel B); 3). single I.P tunicamycin, with thapsagargin (See FIG. 13, Panel C); and 4). thapsagargin+PPG (See FIG. 13, Panel D). In groups 2 and 4, PPG treatment was maintained by daily injection until sacrifice at three days post vehicle/thapsagargin injection. After the completion of this treatment regime, all mice were anaesthetized (isoflourane/oxygen) and sacrificed by decapitation.

While it is not necessary to understand the mechanism of action, it is believed that the histological analysis revealed severe vacuolation, steatosis and acute tubular necrosis with swelling of proximal tubular epithelial cells with pyknotic nuclei and tubules with focal areas of denuded basal lamina. The histological alterations were primarily located in the proximal tubular epithelium. (See FIG. 13 showing images from mice taken from each experimental group. n=5 for each group). These morphological changes were completely ablated in the mice that were induced to accumulated cystathionine by PPG treatment.

### Use of Cystathionine in Treating Neuroblastoma

Neuroblastoma (NB) is the commonest and most deadly solid tumor in children under the age of 5 years and the second most common cause of death after accidents in children. Most children older than 1 year have extensive or metastatic disease at diagnosis, and their prognosis is generally poor. New therapeutic strategies are therefore urgently needed.

### Causes

Neuroblastoma can occur in many areas of the body. It develops from the tissues that form the sympathetic nervous system (the part of the nervous system that controls body functions, such as heart rate and blood pressure, digestion, and levels of certain hormones).

Most neuroblastomas begin in the abdomen in the adrenal gland or next to the spinal cord, or in the chest. They may also start in other areas. Neuroblastomas can spread to the bones (face, skull, pelvis, shoulders, arms, and legs), bone marrow, liver, lymph nodes, skin, and around the eyes (orbits). The cause of the tumor is unknown. Neuroblastoma is most commonly diagnosed in children before age 5. The disorder occurs in approximately 1 out of 100,000 children and is slightly more common in boys. In most patients, the neuroblastoma has already spread when it is first diagnosed.

### Symptoms

The first symptoms are usually fever, a general sick feeling (malaise), and pain. There may also be loss of appetite, weight loss, and diarrhea. Other symptoms depend on the site of the tumor, and may include, but are not limited to: bone pain or tenderness (if the cancer spreads to the bones), difficulty breathing or a chronic cough (if the cancer spreads to the chest), enlarged abdomen (from a large tumor or excess fluid), flushed, red skin, Pale skin and bluish color around the eyes, profuse sweating, rapid pulse (tachycardia).

Brain and nervous system problems may include, but are not limited to: inability to empty the bladder, loss of movement (paralysis) of the hips, legs, or feet (lower extremities), problems with balance, uncontrolled eye movements or leg and feet movements (called opsoclonus-myoconus syndrome, or “dancing eyes and dancing feet”).

### Uses

Cystathionine is not found in the diet and is produced exclusively by the condensation of homocysteine and serine in a reaction catalyzed by cystathionine beta-synthase (CBS) in mammalian biosynthesis of cysteine. To date, no other function has been ascribed to cystathionine. Multiple independent groups have observed massive accumulation of cystathionine and its subsequent excretion in the urine termed cystathioninuria as a frequent and highly specific marker of NB.

Thus, while it is not necessary to understand the mechanism of action, it is believed that in neuroblastoma (NB), patients exhibit cystathioninuria, which has been used as a biomarker for diagnosing NB. The present embodiment contemplates that cystathionine synthesis inhibitor administration may increase tumor cell apoptosis and/or increase the efficacy of chemotherapeutic treatment.

For example, preliminary work has shown that CBS is highly induced in NB and that the next enzyme in the pathway, cystathionine gamma lyase, is not expressed leading to accumulation of cystathionine. Subsequent in vitro and in vivo experiments have shown that cystathionine exerts potent anti-apoptotic effects and that inhibition of the synthesis of this compound could result in increased sensitivity to chemotherapeutic agents in NB.

Further, some embodiments contemplate that the cystathionine accumulated in neuroblastoma has the potential to allow the cells to resist cytotoxic treatments such as chemotherapy and that the use of a CBS inactivating drug might serve to increase chemo sensitization and improve clinical outcome.

### Utilities of Cystathionine

While it is not necessary to understand the mechanism of action, some embodiments contemplate for the first time, direct experimental evidence of a possible function for cystathionine independent of its role as an intermediate in transsulfuration. Previous work has shown that cystathionine can exert protective effects in a mouse model of acetaminophen-induced liver injury by serving as a cysteine prodrug (24). It is believed that the use of PPG in the above experiments, which induces hypercystathionemia by blocking the conversion of cystathionine to cysteine (25) coupled with the observation that cystathionine exerts significant protective effects in a23 cells that are incapable of converting this compound to cysteine, suggest that the protective effects observed in the above experiments are not a result of cystathionine acting as a precursor for cysteine synthesis.

For example, a number of indirect lines of evidence support possible additional physiological roles for either cystathionine or a derivative thereof. In primate brain and CNS, there appears to be an imbalance between the relative activity levels of CBS and CGL leading to an accumulation of cystathionine (26). Similarly, during embryonic development, CBS is expressed in multiple tissues such as the heart and lungs that do not express any detectable CBS in adult tissues (27). A range of data indicates that the next enzyme in the pathway, CGL, is not expressed during early mammalian development. For example, in human liver samples, CGL activity may only be detected in adult tissue whereas activity in fetal, premature and full-term neonatal liver tissue is essentially undetectable (28-30). Therefore, the above data suggests that at certain stages of development and in adult neural tissues, CBS is expressed specifically for the production of cystathionine distinct from its role as an intermediate in cysteine synthesis.

While it is not necessary to understand the mechanism of action, it is believed that the above data suggests, using both tissue culture and whole animal models strongly point to a role for cystathionine in protecting against ER stress induced tissue damage and cell death. However, this interpretation must be qualified somewhat as previous research has shown that in addition to the transsulfuration pathway, there are other metabolic fates for cystathionine, the biochemistry of which is only partially understood. Previous investigations have observed that inducing elevated cystathionine with PPG in rats also results in a detectable increase in the cystathionine metabolites perhydro-1,4-thiazepine-3,5-dicarboxylic acid, cystathionine mono-oxo acids [S-(3-oxo-3-carboxy-n-propyl)cysteine and S-(2-oxo-2-carboxyethyl)homocysteine], cystathionine ketimines, cystathionine sulfoxide and N-acetylcystathionine sulfoxide. The level of these compounds increase in direct proportion to the scale of the accumulation of cystathionine (31). The increase in the levels of these cystathionine metabolites is not an artifact of the PPG compound itself as all of these metabolites been identified previously at elevated levels in the urine of patients with cystathioninuria due to mutational inactivation of the human CGL gene (32). This is a generic concern in interpreting protective effects of a compound where the biochemistry, regulation and tissue distribution of its metabolism are incompletely understood but at this stage, it cannot unequivocally be ruled out that one or more of cystathionine metabolites may be responsible for the protective effects observed in this study. Thus, additional work might be required to see if any of these cystathionine metabolites are capable of exerting cytoprotective effects.

For example, CBSDH is unique among the genetic homocystinurias in that in addition to elevated Hcy, methionine, AdsoMet and AdoHcy, it also incurs a concomitant abolition of cystathionine and cysteine synthesis. While cysteine can be obtained from the diet, CBSDH acts to effectively remove cystathionine. The finding that cystathionine exerts cytoprotective effects raises the possibility that the abolition of its synthesis might contribute to pathogenesis in CBSDH. The observation that the HO mouse model of CBSDH exhibits a hypercoagulative phenotype in the presence of 4-fold higher than normal levels of cystathionine and the fact that increased risk of thromboembolic complications is a common feature of all of the genetic homocystinurias indicates that the abolition of cystathionine synthesis is unlikely to be contributing to this aspect of pathogenesis.

The connective tissue defects of CBSDH are unique among the genetic homocystinurias. Because of the strong similarity between these sequelae and those observed in the fibrillinopathy Marfan syndrome, the leading candidate mechanism for connective tissue disturbances in CBSDH is impairment of the folding/function of fibrillin-1. This protein is very cysteine rich, much of which appears in the free reactive sulfhydryl form. The functional significance of these vulnerable cysteines is underscored by the fact that many of the fibrillin-1 mutations found in Marfan syndrome delete or alter the spacing of these residues (33). Because of this high cysteine content it is likely that the folding pathway and assembly of this protein is relatively complicated in order to prevent the inappropriate juxtaposition of cysteine residues and subsequent formation of erroneous disulfide bonds. Consequently, fibrillin-1 may be particularly vulnerable to the protein misfolding that is typically associated with ER stress. Recent work has suggested homocysteinylation of cysteine residues in fibrillin-1 as a mechanism for impairing folding and inducing fibrillinopathy in CBSDH (34,35). It is not known yet exactly which mechanism/s by which cystathionine is exerting protective effects but it is believed that cystathionine may protect against ER stress by serving as a chemical chaperone and could conceivably assist with the correct folding of fibrillin-1 in the presence of elevated Hcy.

The mechanisms that underlie mental retardation in CBSDH are unknown but as described above, CBS and CGL expression in the mammalian brain appeared to be specifically tailored towards the accumulation of cystathionine. Enhanced levels of ER stress are implicated in various neuropathological conditions, for example, and not meant to be limiting brain ischemia and excitotoxicity in neurons (36) and neurodegenerative conditions such as Parkinson's disease (37). While it is not necessary to understand the mechanism of action, it is believed that it may be possible that cystathionine specifically accumulates in the normal mammalian brain to serve as a cytoprotectant and that the abolition of its synthesis contributes to mental retardation in CBSDH by increasing sensitivity of neural tissues to the toxic insult of elevated Hcy and/or derivatives thereof.

The mechanism by which cystathionine exerts its protective effect is currently unknown but our findings offer a number of clues and raise the possibility that the protective effects of this compound may not be limited to ER stress. Apoptosis is a point of convergence of multiple distinct pathways of cellular perturbation including oxidative stress, inflammatory factors such as TNF-alpha and ER stress. While it is not necessary to understand the mechanism of action, it is believed that cystathionine may be capable of blocking tunicamycin mediated tissue injury and apoptosis without affecting induction levels of the UPR argues and that its protective effects occur downstream of GADD153 and may therefore be capable of exerting a more generalized effect against the induction of tissue damage and apoptosis. The process of apoptosis can be functionally divided into two distinct phases, induction and execution. A key a point of convergence of the many different apoptogenic stimuli in the induction phase has been shown to be the specific extrusion of cellular thiols including the antioxidant glutathione (GSH) in the reduced form prior to any plasma membrane leakage (38-40). Cystathionine has been shown to be able to inhibit apoptosis independent of its role as a cysteine donor compound by inhibiting the specific sinusoidal type carrier mediated efflux of GSH in two different cell lines. The ability of cystathionine to inhibit apoptosis indicated that this compound may exert its protective role at an early step of the induction phase (i.e., for example, before any irreversible involution of cellular structure occurs). Given the almost normal appearance of the liver and kidney in both HO and PPG treated hypercystathionemic mice treated with tunicamycin and the almost complete lack of cell death in the tissue culture studies, it is believed that cystathionine may exert a protective effect in the induction phase of apoptosis in these models. If thiol extrusion is a point of conversion for multiple apoptogenic stimuli it is quite possible that cystathionine could exert protective effects against multiple intracellular disturbances in addition to ER stress.

While it is not necessary to understand the mechanism of action, it is believed that, in addition to CBSDH, elevated cystathionine may promote cell survival in the face of cytotoxic challenge. Neuroblastoma is the commonest and most deadly solid tumor in children under the age of 5 years (50% of cases before 2 years, 90% before 5) (41,42). Most children older than 1 year have extensive or metastatic disease at diagnosis, and their prognosis is generally poor. Massive accumulation of cystathionine and subsequent cystathioninuria has been observed as a frequent and highly specific marker for neuroblastoma by multiple independent groups (43-46). While it is not necessary to understand the mechanism of action, it is believed that the data indicates that the cystathionine accumulated in neuroblastoma has the potential to allow the cells to resist cytotoxic treatments such as chemotherapy and that the use of a CBS inactivating drug might serve to increase chemosensitization and improve clinical outcome.

Thus, while it is not necessary to understand the mechanism of action, it is believed that cystathionine in addition to serving as an intermediate in transsulfuration, exerts significant cytoprotective effects against ER stress mediated tissue injury without modulating expression of the UPR and that abolition of its synthesis has the potential to contribute to pathogenesis in CBSDH. While it is not necessary to understand the mechanism of action, it is believed that collectively, the above data suggests that cystathionine may have significant therapeutic potential in disease states where ER stress (Some publications discussing ER stress include, but are not limited to, Cunard et al., “The Endoplasmic Reticulum Stress Response and Diabetic Kidney Disease,” Am J Physiol Renal Physiol, May 2011, 300(5):F105-61; Tabas et al., “Integrating the Mechanisms of Apoptosis Induced by Endoplasmic Reticulum Stress,” Nat Cell Biol, 2011 13(3):184-90; and Thomas et al., “Diabetes As A Disease of Endoplasmic Reticulum Stress,” Diabetes Metab Res Rev., 2010 26(8):611-21; each of which is hereby incorporated by reference in its entirety) is implicated as a pathogenic factor.

### Drug Delivery Systems

In some embodiments, the present invention contemplates several drug delivery systems that provide for roughly uniform distribution, have controllable rates of release. A variety of different media are described below that are useful in creating drug delivery systems. It is not intended that any one medium or carrier is limiting to the present invention. Note that any medium or carrier may be combined with another medium or carrier; for example, in one embodiment a polymer microparticle carrier attached to a compound may be combined with a gel medium.

Carriers or mediums contemplated by this invention comprise a material selected from the group comprising gelatin, collagen, cellulose esters, dextran sulfate, pentosan polysulfate, chitin, saccharides, albumin, fibrin sealants, synthetic polyvinyl pyrrolidone, polyethylene oxide, polypropylene oxide, block polymers of polyethylene oxide and polypropylene oxide, polyethylene glycol, acrylates, acrylamides, methacrylates including, but not limited to, 2-hydroxyethyl methacrylate, poly(ortho esters), cyanoacrylates, gelatin-resorcin-aldehyde type bioadhesives, polyacrylic acid and copolymers and block copolymers thereof.

One embodiment of the present invention contemplates a drug delivery system comprising therapeutic agents as described herein.

### Microparticles

One embodiment of the present invention contemplates a medium comprising a microparticle. Preferably, microparticles comprise liposomes, nanoparticles, microspheres, nanospheres, microcapsules, and nanocapsules. Preferably, some microparticles contemplated by the present invention comprise poly(lactide-co-glycolide), aliphatic polyesters including, but not limited to, poly-glycolic acid and poly-lactic acid, hyaluronic acid, modified polysacchrides, chitosan, cellulose, dextran, polyurethanes, polyacrylic acids, psuedo-poly(amino acids), polyhydroxybutrate-related copolymers, polyanhydrides, polymethylmethacrylate, poly(ethylene oxide), lecithin and phospholipids.

### Liposomes

One embodiment of the present invention contemplates liposomes capable of attaching and releasing therapeutic agents described herein. Liposomes are microscopic spherical lipid bilayers surrounding an aqueous core that are made from amphiphilic molecules such as phospholipids. For example, a liposome may trap a therapeutic agent between the hydrophobic tails of the phospholipid micelle. Water soluble agents can be entrapped in the core and lipid-soluble agents can be dissolved in the shell-like bilayer. Liposomes have a special characteristic in that they enable water soluble and water insoluble chemicals to be used together in a medium without the use of surfactants or other emulsifiers. Liposomes can form spontaneously by forcefully mixing phosopholipids in aqueous media. Water soluble compounds are dissolved in an aqueous solution capable of hydrating phospholipids. Upon formation of the liposomes, therefore, these compounds are trapped within the aqueous liposomal center. The liposome wall, being a phospholipid membrane, holds fat soluble materials such as oils. Liposomes provide controlled release of incorporated compounds. In addition, liposomes can be coated with water soluble polymers, such as polyethylene glycol to increase the pharmacokinetic half-life. One embodiment of the present invention contemplates an ultra-high-shear technology to refine liposome production, resulting in stable, unilamellar (single layer) liposomes having specifically designed structural characteristics. These unique properties of liposomes, allow the simultaneous storage of normally immiscible compounds and the capability of their controlled release.

In some embodiments, the present invention contemplates cationic and anionic liposomes, as well as liposomes having neutral lipids. Preferably, cationic liposomes comprise negatively-charged materials by mixing the materials and fatty acid liposomal components and allowing them to charge-associate. Clearly, the choice of a cationic or anionic liposome depends upon the desired pH of the final liposome mixture. Examples of cationic liposomes include lipofectin, lipofectamine, and lipofectace.

One embodiment of the present invention contemplates a medium comprising liposomes that provide controlled release of at least one therapeutic agent. Preferably, liposomes that are capable of controlled release: i) are biodegradable and non-toxic; ii) carry both water and oil soluble compounds; iii) solubilize recalcitrant compounds; iv) prevent compound oxidation; v) promote protein stabilization; vi) control hydration; vii) control compound release by variations in bilayer composition such as, but not limited to, fatty acid chain length, fatty acid lipid composition, relative amounts of saturated and unsaturated fatty acids, and physical configuration; viii) have solvent dependency; iv) have pH-dependency and v) have temperature dependency.

The compositions of liposomes are broadly categorized into two classifications. Conventional liposomes are generally mixtures of stabilized natural lecithin (PC) that may comprise synthetic identical-chain phospholipids that may or may not contain glycolipids.

Special liposomes may comprise: i) bipolar fatty acids; ii) the ability to attach antibodies for tissue-targeted therapies; iii) coated with materials such as, but not limited to lipoprotein and carbohydrate; iv) multiple encapsulation and v) emulsion compatibility.

Liposomes may be easily made in the laboratory by methods such as, but not limited to, sonication and vibration. Alternatively, compound-delivery liposomes are commercially available. For example, Collaborative Laboratories, Inc. are known to manufacture custom designed liposomes for specific delivery requirements.

### Microspheres, Microparticles and Microcapsules

Microspheres and microcapsules are useful due to their ability to maintain a generally uniform distribution, provide stable controlled compound release and are economical to produce and dispense. Preferably, an associated delivery gel or the compound-impregnated gel is clear or, alternatively, said gel is colored for easy visualization by medical personnel.

Microspheres are obtainable commercially (Prolease®, Alkerme's: Cambridge, Mass.). For example, a freeze-dried medium comprising at least one therapeutic agent is homogenized in a suitable solvent and sprayed to manufacture microspheres in the range of 20 to 90 μm. Techniques are then followed that maintain sustained release integrity during phases of purification, encapsulation and storage. Scott et al., Improving Protein Therapeutics With Sustained Release Formulations, Nature Biotechnology, Volume 16:153-157 (1998).

Modification of the microsphere composition by the use of biodegradable polymers can provide an ability to control the rate of therapeutic agent release. Miller et al., Degradation Rates of Oral Resorbable Implants {Polylactates and Polyglycolates: Rate Modification and Changes in PLA/PGA Copolymer Ratios, J. Biomed. Mater. Res., Vol. II:711-719 (1977).

Alternatively, a sustained or controlled release microsphere preparation is prepared using an in-water drying method, where an organic solvent solution of a biodegradable polymer metal salt is first prepared. Subsequently, a dissolved or dispersed medium of a therapeutic agent is added to the biodegradable polymer metal salt solution. The weight ratio of a therapeutic agent to the biodegradable polymer metal salt may for example be about 1:100000 to about 1:1, preferably about 1:20000 to about 1:500 and more preferably about 1:10000 to about 1:500. Next, the organic solvent solution containing the biodegradable polymer metal salt and therapeutic agent is poured into an aqueous phase to prepare an oil/water emulsion. The solvent in the oil phase is then evaporated off to provide microspheres. Finally, these microspheres are then recovered, washed and lyophilized. Thereafter, the microspheres may be heated under reduced pressure to remove the residual water and organic solvent.

Other methods useful in producing microspheres that are compatible with a biodegradable polymer metal salt and therapeutic agent mixture are: i) phase separation during a gradual addition of a coacervating agent; ii) an in-water drying method or phase separation method, where an antiflocculant is added to prevent particle agglomeration and iii) by a spray-drying method.

In one embodiment, the present invention contemplates a medium comprising a microsphere or microcapsule capable of delivering a controlled release of a therapeutic agent for a duration of approximately between 1 day and 6 months. In one embodiment, the microsphere or microparticle may be colored to allow the medical practitioner the ability to see the medium clearly as it is dispensed. In another embodiment, the microsphere or microcapsule may be clear. In another embodiment, the microsphere or microparticle is impregnated with a radio-opaque fluoroscopic dye.

Controlled release microcapsules may be produced by using known encapsulation techniques such as centrifugal extrusion, pan coating and air suspension. Such microspheres and/or microcapsules can be engineered to achieve desired release rates. For example, Oliosphere® (Macromed) is a controlled release microsphere system. These particular microsphere's are available in uniform sizes ranging between 5-500 μm and composed of biocompatible and biodegradable polymers. Specific polymer compositions of a microsphere can control the therapeutic agent release rate such that custom-designed microspheres are possible, including effective management of the burst effect. ProMaxx® (Epic Therapeutics, Inc.) is a protein-matrix delivery system. The system is aqueous in nature and is adaptable to standard pharmaceutical delivery models. In particular, ProMaxx® are bioerodible protein microspheres that deliver both small and macromolecular drugs, and may be customized regarding both microsphere size and desired release characteristics.

In one embodiment, a microsphere or microparticle comprises a pH sensitive encapsulation material that is stable at a pH less than the pH of the internal mesentery. The typical range in the internal mesentery is pH 7.6 to pH 7.2. Consequently, the microcapsules should be maintained at a pH of less than 7. However, if pH variability is expected, the pH sensitive material can be selected based on the different pH criteria needed for the dissolution of the microcapsules. The encapsulated compound, therefore, will be selected for the pH environment in which dissolution is desired and stored in a pH preselected to maintain stability. Examples of pH sensitive material useful as encapsulants are Eudragit® L-100 or S-100 (Rohm GMBH), hydroxypropyl methylcellulose phthalate, hydroxypropyl methylcellulose acetate succinate, polyvinyl acetate phthalate, cellulose acetate phthalate, and cellulose acetate trimellitate. In one embodiment, lipids comprise the inner coating of the microcapsules. In these compositions, these lipids may be, but are not limited to, partial esters of fatty acids and hexitiol anhydrides, and edible fats such as triglycerides. Lew C. W., Controlled-Release pH Sensitive Capsule and Adhesive System and Method. U.S. Pat. No. 5,364,634 (herein incorporated by reference).

In one embodiment, the present invention contemplates a microparticle comprising a gelatin, or other polymeric cation having a similar charge density to gelatin (i.e., poly-L-lysine) and is used as a complex to form a primary microparticle. A primary microparticle is produced as a mixture of the following composition: i) Gelatin (60 bloom, type A from porcine skin), ii) chondroitin 4-sulfate (0.005%-0.1%), iii) glutaraldehyde (25%, grade 1), and iv) 1-ethyl-3-(3-dimethylaminopropyl)-carbodiimide hydrochloride (EDC hydrochloride), and ultra-pure sucrose (Sigma Chemical Co., St. Louis, Mo.). The source of gelatin is not thought to be critical; it can be from bovine, porcine, human, or other animal source. Typically, the polymeric cation is between 19,000-30,000 daltons. Chondroitin sulfate is then added to the complex with sodium sulfate, or ethanol as a coacervation agent.

Following the formation of a microparticle, a therapeutic agent is directly bound to the surface of the microparticle or is indirectly attached using a “bridge” or “spacer”. The amino groups of the gelatin lysine groups are easily derivatized to provide sites for direct coupling of a compound. Alternatively, spacers (i.e., linking molecules and derivatizing moieties on targeting ligands) such as avidin-biotin are also useful to indirectly couple targeting ligands to the microparticles. Stability of the microparticle is controlled by the amount of glutaraldehyde-spacer crosslinking induced by the EDC hydrochloride. A controlled release medium is also empirically determined by the final density of glutaraldehyde-spacer crosslinks.

In one embodiment, the present invention contemplates microparticles formed by spray-drying a composition comprising fibrinogen or thrombin with a therapeutic agent. Preferably, these microparticles are soluble and the selected protein (i.e., fibrinogen or thrombin) creates the walls of the microparticles. Consequently, the therapeutic agents are incorporated within, and between, the protein walls of the microparticle. Heath et al., Microparticles And Their Use In Wound Therapy. U.S. Pat. No. 6,113,948 (herein incorporated by reference). Following the application of the microparticles to living tissue, the subsequent reaction between the fibrinogen and thrombin creates a tissue sealant thereby releasing the incorporated compound into the immediate surrounding area.

One having skill in the art will understand that the shape of the microspheres need not be exactly spherical; only as very small particles capable of being sprayed or spread into or onto a surgical site (i.e., either open or closed). In one embodiment, microparticles are comprised of a biocompatible and/or biodegradable material selected from the group consisting of polylactide, polyglycolide and copolymers of lactide/glycolide (PLGA), hyaluronic acid, modified polysaccharides and any other well-known material.

### Pharmaceutical Compositions

In some embodiments, the present invention contemplates pharmaceutical compositions (e.g., comprising the compounds described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of some embodiments include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present embodiments, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present embodiments may be formulated into any of many possible dosage forms such as, but not limited to, tablets, capsules, liquid syrups, soft gels, suppositories, and enemas. The compositions of the present embodiments may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the compound is administered in maintenance doses, ranging from 0.01 μg to 100 g per kg of body weight, once or more daily, to once every 20 years.

### EXPERIMENTAL

The following examples serve to illustrate certain preferred embodiments and aspects of the present invention and are not to be construed as limiting the scope thereof.

Statistical Analysis: All data are presented as means±SD and were compared using the unpaired Student's t test. A P value of less than 0.05 was considered statistically significant. In the graphed data *, **, and *** denote P values of <0.05., 0.01 and 0.001 respectively.

### Example I

### Chemicals and Reagents

Unless otherwise stated, all chemicals were obtained from Sigma (St. Louis, Mo.). All reagents for mammalian tissue culture were obtained from Life Sciences Technologies (Rockville, Md.) except for fetal calf serum, which was obtained from HyClone (Logan, Utah).

### Example II

### Media and Mammalian Cell Culture

CBS and CGL positive HepG2 hepatocellular carcinoma cells and human embryonal kidney 293AD cells were obtained from the American Type Culture Collection (Manassas, Va.) and were cultured as described previously (6). CBS and CGL negative Chinese hamster fibroblast a23 cells were obtained from the University of Colorado Intellectual Developmental Disabilities Research Center central collection and were cultured as described previously (6).

### Example III

### Tunicamycin Treatment of Cultured Cells and Assessment of Cell Death

HepG2, 293AD and a23 cells were seeded in triplicate at ˜75% confluence and were pre-treated with either propargylglycine (PPG) (5 mM) or cystathionine (5 mM) in complete media (DMEM, 10% FBS). After 24 hours, fresh media with 1% FBS was added to each well. Cells were then further treated with tunicamycin (either 5 or 10 ug/ml) for 18 hr treatment. Cells were subsequently photographed under phase-contrast with a Zeiss Axiovert 25 microscope for evidence of dead floating cells. To quantify cell death the media supernatant was collected for LDH release assay using a kit from Roche Applied Science (Cat.No.11 644 793 001) according to the manufacturer's standard protocol where cytotoxicity (%)=((experimental value-low control/(high control-low control))×100.

### Example IV

### Animal Experiments and Tissue Sample Analysis

All animal experiments were pre-approved by the University of Colorado Health Sciences Center institutional animal care and use committee. Male and female HO mice (6 to 10 weeks old) were generated as described previously (5). Male and female, C57BL/6J mice (7-11 wks) were used as wild type (WT) controls and were bred in house and housed in colony cages. Both strains were maintained on a 12-hour light/dark cycle and had free access to food and water.

### Example V

### Animal Treatments

Propensity for hepatic steatosis was examined in HO and wild type control mice using a methionine-choline deficient amino acid defined and iron supplemented diet (#518810 Dyets Inc., Bethlehem, Pa.)) and an isocaloric amino acid defined Lombardi choline sufficient diet with methionine control diet (518754, Dyets Inc., Bethlehem, Pa.) for 19 days as described previously (7). Experimental hypercystathionemia was induced by IP injection of the CGL inactivator PPG (50 mg·day-1·kg-1) as described previously (8). ER stress was induced in mice by a single IP injection with tunicamycin at 0.5 mg/kg body weight (volume <=1.0 ml). Control mice were sham treated by injection with an equal volume of PBS (9,10).

### Example VI

### Thiols and Methionine Cycle Metabolites

Determination of plasma levels of methionine cycle metabolites was performed as described previously (11).

### Example VII

### Histological Examination of Tissues and Assessment of Hepatopathy

Tissues were immersion-fixed overnight in 4% paraformaldehyde in PBS (pH 7.3). Paraffin embedded sections (5 μm) were stained for examination with hematoxylin and eosin (H and E). Cells undergoing apoptosis were identified by labeling their DNA 3′-OH nick ends using a variant of terminal deoxynucleotidyl transferase-mediated dUTP nick end-labeling (TUNEL) staining. Staining was performed according to the manufacturer's instruction with ApopTag® Peroxidase in Situ Apoptosis Detection Kit (Millipore, Billerica, Mass.). Tissue sections were deparaffinized with xylene and washed in succession with different concentrations of ethanol (absolute, 95% and 70%). Tissue sections were then digested with proteinase K (20 μg/ml) for 15 min. Endogenous peroxidase activity was quenched with 3% H₂O₂ in PBS for 5 min. The slides were immersed in terminal deoxynucleotidyl transferase (TdT) buffer containing digoxigenin-labeled nucleotides at 37° C. for 1 h. After washing, anti-digoxigenin-peroxidase was added to cover the slides and incubated in a humid chamber at room temperature for 30 min. After washing with PBS, the slides were stained with diaminobenzidine and then counterstained with 1% methyl green. Light microscopy was performed to quantify the apoptotic cells, by counting cells in ten randomly selected fields (×100 magnification) of each slide with the following formula: number of apoptotic cells/total number of cells counted×100.

A party unaware of the animal's genotype or treatment group performed both histological analysis, of stained slides and TUNEL staining. Liver injury was assessed by determining plasma levels of alanine aminotransferase (ALT) activity using an enzyme-coupled assay with lactic dehydrogenase (LDH) as described previously (12) Liver lipid was extracted using the procedure of Bligh and Dyer (13) and after evaporation of the organic solvent, the triacylglycerol content of each sample was measured in duplicate using an enzymatic method (Sigma-Aldrich).

### Example VIII

### Immmunoblot Analysis

Immunoblot analysis of total cell lysates was performed as described previously (4,14). Briefly, following incubation with the appropriate primary and horseradish peroxidase-conjugated secondary antibodies (Affinity Biologicals, Ancaster, ON, Canada) membranes were developed using the Renaissance® Western Blot Chemiluminescence Reagent Kit, (Perkin Elmer, Woodbridge, ON, Canada). Proteins were transferred on a nitrocellulose membrane, and probed with anti-KDEL monoclonal antibody, which recognizes Hsp70, GRP78 and GRP94 that was purchased from Stressgen Biotechnologies (SPA-827) or anti GADD153 (Santa Cruz Biotech #SC-7351). Anti-β-Actin monoclonal antibody (Sigma) was used as a loading control.

### Example IX

### Preparation of Total RNA and Northern Blot Analysis

Total RNA was isolated from 293AD cells using the RNeasy® total RNA kit (Qiagen, Santa Clarita, Calif.) and resuspended in diethylpyrocarbonate (DEPC)-treated water. Quantification and purity of the RNA was assessed by A260/A280 absorption, and RNA samples with ratios greater than 1.6 were stored at 70° C. for further analysis. Total RNA (10 μg/lane) was fractionated on 2.2 mol/L formaldehyde/1.2% agarose gels and transferred overnight onto Zeta-Probe® GT nylon membranes (Bio-Rad, Toronto, Canada) in 10×SSC. The RNA was cross-linked to the membrane using a UV crosslinker (PDI Bioscience, Toronto, Canada) before hybridization. Specific probes were generated by labeling cDNA fragments with [−32P]dCTP (NEN) using a random primed DNA labeling kit (Boehringer Mannheim, Laval, Canada). The cDNA probe for GADD153 (no. AA627477) was obtained from Genome Systems (St Louis, Mo.). The cDNA probe used for GRP78 is a full length cDNA clone that has been described previously (4). After overnight hybridization at 43° C., the membranes were washed as described by the manufacturer, exposed to x-ray film, and subjected to autoradiography.

### Example X

### HO CBSDH Mice Exhibit Significantly Attenuated Progression of Diet-Induced Hepatic Steatosis

To date, all cbs null mouse models of CBSDH have been found to incur liver injury and a high degree of neonatal lethality (2,3,15). Those cbs null mice that survive the neonatal period invariably incur profound hepatic steatosis (2,3). Despite having essentially identical levels of plasma and tissue Hcy as the cbs null models, the HO CBSDH mice do not exhibit any discernible hepatic steatosis (5) leading us to speculate that the elevated cystathionine exhibited by HO mice might be exerting protective effects against aberrant hepatic lipid accumulation. To investigate this possibility, the relative degree of hepatic lipid accumulation in HO and wild type control mice fed a methionine and choline deficient (MCD) diet was determined. This diet rapidly induces hepatic steatosis as methionine and choline are lipotropic precursors of phosphatidylcholine synthesis and their exclusion from the diet inhibits the assembly of very low density lipoprotein (VLDL) causing impairment of the secretion of triglycerides and free fatty acids from hepatocytes (16).

One group of HO mice and one group of WT control mice (n=5 for each) were put on an MCD diet. Control groups of both genotypes (n=5) were put on a chow diet that matched the composition of the MCD diet except that it contained normal levels of choline and methionine. All groups were kept on their respective diets for a total of 17 days. At the end of the trial, the mice were sacrificed and the livers of these animals were examined. A party unaware of the animals' genotype or treatment group performed histological analysis. No hepatic steatosis was observed in either of the control groups (data not shown). Histological examination of the livers after H and E staining found that as expected, the MCD diet induced severe macrovesicular hepatic steatosis, ballooning degeneration of hepatocytes and multiple inflammatory foci in the WT mice. These sequelae were greatly attenuated in the HO CBSDH mice (FIG. 2, Panel A). To quantitate the development of hepatic steatosis induced by the MCD diet, the triglyceride content in the livers of the MCD fed HO and WT mice and in the animals fed the control diet (FIG. 2, Panel B) were measured. The lipid levels of both the HO and WT animals on the control chow diet did not differ significantly from each other (p=0.707). Compared to the relevant genotype chow controls, the MCD diet induced an approximate 6-fold increase in the hepatic triglyceride levels of WT animals but only a 2-fold increase in HO mice. This observation is consistent with our histological analysis and collectively, these data support the possibility that elevated cystathionine can exert hepatoprotective effects.

### Example XI

### HO Mice Exhibit Ablated Hepatic and Renal Injury after Tunicamycin Treatment

The ER is the primary site for the synthesis, folding and modification of proteins in the eukaryotic cell and exposure of cells to conditions such as inhibition of protein glycosylation, nutrient/oxygen deprivation or perturbation of Ca²⁺ homeostasis, results in the accumulation of unfolded proteins causing ER stress (17). Previous work has indicated that the MCD diet induces hepatic steatosis at least in part by inducing ER stress (7). Similarly, previous work has suggested that the steatosis observed in cbs null mice is directly related to the action of elevated Hcy inducing ER stress causing an alteration of serum response element binding protein 1 (SREBP1) function that subsequently results in dysfunctional lipid metabolism (4). The ability of the HO mice to resist hepatic steatosis in the presence of an MCD diet and our previous observation of distended ER in the steatotic cbs null mice but not the HO mice raises the possibility that elevated cystathionine in HO mice is exerting its hepatoprotective effects by blocking the induction of ER stress.

To examine this possibility, a tunicamycin mediated model of ER stress induced tissue injury on groups of HO and WT mice was use. The nucleoside antibiotic, tunicamycin, produced by the actinomycete, Streptomyces lysosuperifcus, causes ER stress by inhibiting UDP-N-acetylgluco samine:dolichol phosphate N-acetylglucosamine-1-P transferase blocking protein N-glycosylation. Previous work has shown that a single sub-lethal intraperitoneal injection with 0.5 mg/kg body weight of tunicamycin results in a reproducible pattern of lassitude, lack of grooming, weight loss, renal injury and hepatic steatosis that peaks between day 4 and 5 post-injection (9,10). Groups of WT and HO (n=5 for all groups) mice were given either a single intra-peritoneal injection of tunicamycin or sham injected with an identical volume of the vehicle solution PBS. All mice treated with tunicamycin became markedly inappetant shortly after injection and marked weight loss was clearly noticeable and progressive during the course of the experiment. Three days after the administration of treatment, mice were sacrificed and tissues were extracted and processed for histological analysis. In the sham injected control animals no evidence of tissue injury or steatosis was observed (data not shown). In the WT control mice as expected, significant weight loss, lassitude and altered fur was observed. Additionally, it was observed that the livers of the tunicamycin treated wild type animals were severely enlarged and yellowish in color. Histologically, H and E staining revealed a clearly discernible pattern of periportal hepatocellular damage, severe vacuolation of hepatocytes was observed much of which appeared to result from lipid accumulation. Single, condensed, rounded or ovoid cytoplasmic bodies, apparently derived from degenerating hepatocytes, were scattered throughout the liver, but especially prevalent in the periportal region (FIG. 3).

The most severe injury was observed in the kidney where significant enlargement and accumulation of fat around the organ. Histological analysis revealed severe vacuolation, steatosis and acute tubular necrosis with swelling of proximal tubular epithelial cells with pyknotic nuclei and tubules with focal areas of denuded basal lamina. The histological alterations were primarily located in the proximal tubular epithelium (FIG. 3). These morphological changes are very similar to those described in mice and other species following tunicamycin injection (9,18,19).

HO mice treated with tunicamycin incurred essentially identical weight loss, lassitude and altered grooming as the WT mice but strikingly, did not exhibit any significant liver enlargement with only minimal hepatic steatosis and no discernible alteration of renal morphology (FIG. 3). These data are consistent with the possibility that elevated cystathionine acts to protect against ER stress mediated tissue injury in both the liver and kidney.

### Example XII

### Daily Injection with the CGL Inactivating Compound Propargylglycine Cause Temporary Hypercystathionemia in Normal Mice

Although the data described above indirectly supports the possibility that cystathionine exerts tissue protective effects at least in part by blocking the pathogenic consequences of prolonged ER stress, there are multiple biochemical differences between the HO mice and WT mice (3,5). In order to provide more direct evidence of a protective role for cystathionine, it was proposed to induce elevated cystathionine in normal mice in order to examine its protective properties in isolation from the elevated Hcy, methionine, AdoMet, AdoHcy and decreased cysteine that is observed in the HO mice.

Inducing elevated cystathionine in normal mice is complicated by the fact that this compound has relatively low solubility in water and is rapidly excreted in the urine of mice. Previous work by Abeles and Walsh synthesized and tested a range of acetylenic substrates capable of irreversible inactivation of CGL. I.P. injection of one of these inactivators D,L-2-amino-4-pentynoic acid also known as propargylglycine (PPG) has been shown to rapidly inactivate hepatic CGL activity and results in a concomitant accumulation of cystathionine within one hour that lasts for approximately one day (20). For our purposes this method has the additional benefit of allowing us to investigate the possible protective effects of elevated cystathionine in isolation from its role as a cysteine donor. To our knowledge, the longevity of this cystathionine accumulation with repeated injections of PPG over time in mice has not been examined. Daily injections of PPG to WT mice were given (n=7) each morning over a period of 4 days. Plasma samples were taken from these mice in the evening of each day by non-lethal tail bleed and the levels of cystathionine, Hcy, methionine and cysteine were determined (FIG. 4). It was observed that inactivation of CGL was accompanied by a 56-fold increase in the plasma level of cystathionine on day 1. This maximal accumulation persisted for the second day and was reduced to 30-fold by day 3. By day 4 of the experiment, the level of cystathionine accumulation was reduced to 8-fold compared to untreated animals indicating that despite daily re-administration of this compound, the effect of PPG diminishes over time. Blocking transsulfuration also induced a relatively mild elevation in total Hcy (tHcy) and methionine that declined back to normal as the efficacy of the PPG treatment diminished. As expected, blocking transsulfuration via PPG treatment resulted in a concomitant decrease in plasma cysteine levels during the first and second days of PPG treatment. Cysteine levels subsequently returned to near normal levels as the effects of the PPG treatment diminished. These experiments allowed us to define an optimal time frame for examining the possible protective effects of PPG mediated cystathionine accumulation in WT mice. The effect of PPG on liver health was assessed by determining ALT values before PPG treatment and at each subsequent time point studied. PPG treatment did not induce any significant increase in plasma ALT levels at any of the time points analyzed indicating that this treatment is relatively benign over the time period studied (data not shown).

### Example XIII

### Elevated Cystathionine in WT Mice Protects Against Hepatic and Renal Damage Mediated by the ER Stress Inducing Agent Tunicamycin

In order to investigate the effects of elevated cystathionine upon the response to ER stress, wild type mice (n=6) were exposed to a single sub-lethal intraperitoneal injection of tunicamycin in the presence and absence of the CGL inactivating compound PPG. WT mice were randomly assigned to one of four treatment groups (n=6 for each group): 1) control, IP injected with PBS; 2) I.P. injection with PPG; 3) single I.P tunicamycin, with tunicamycin and 4) tunicamycin+PPG. In groups 2 and 4, PPG treatment was maintained by daily injection until sacrifice at three days post vehicle/tunicamycin injection. After the completion of this treatment regime, all mice were anaesthetized (isoflourane/oxygen) and sacrificed by decapitation, and blood samples were collected for plasma analysis. Livers and kidneys were removed, weighed and then samples were either immersion-fixed overnight in 4% paraformaldehyde in PBS (pH 7.3) for subsequent histological analysis or were snap frozen in liquid nitrogen and examined for morphological, histological and biochemical analyses.

The livers and kidneys of the sham injected and PPG alone control groups were normal in size and appearance while the livers from the animals treated solely with tunicamycin were enlarged, yellowish and fatty in appearance (FIG. 5, Panel A) Similarly, the kidneys of these animals were also swollen and covered in fat (FIG. 6, Panel A). This enlargement was reflected in increased livers and kidney weight compared to the controls (FIG. 5, Panel B; FIG. 6, Panel B). Determination of triglyceride content in the liver and kidneys found a highly significant increase in the liver (P<0.001) and kidneys (p<0.001) of the animals treated solely with tunicamycin compared to the sham and PPG control groups confirming the induction of hepatic and renal steatosis by this toxin (FIG. 5, Panel C; FIG. 6, Panel C). The induction of significant liver injury by tunicamycin treatment was also confirmed by determination of plasma ALT levels. (FIG. 5, Panel D). Strikingly, livers and kidneys from the hypercystathionemic mice that were treated with tunicamycin appeared ostensibly normal and did not differ significantly from the control groups either in liver weight, triglyceride levels or plasma ALT levels.

Histological analysis revealed that the sham injection and PPG control groups showed no significant morphological alteration while the tunicamycin alone group incurred severe morphological changes in the liver and kidney that were essentially identical to those described above. These morphological changes were effectively ablated in the mice that were induced into hypercystathionemia by PPG treatment during the tunicamycin treatment (FIG. 7, Panels A-B). To further assess the protective effects of cystathionine, tissues of all of these mice were analyzed for evidence of apoptosis using Tunel staining. No apoptosis was observed in either of the control groups. In the tunicamycin treated animals the most affected organ was the kidney. In this tissue, the proximal tubule epithelium in particular showed clear evidence of apoptotic cell death (FIG. 7, Panel C). No apoptotic cells were observed in the kidneys of tunicamycin treated mice that had been pretreated with PPG. Collectively, these results indicate that cystathionine can exert significant protective effects against ER stress mediated lipid accumulation, tissue injury and apoptotic cell death.

### Example XIV

### PPG Induced Hypercystathionemia does not Alter Induction of the Unfolded Protein Response in Mice

Eukaryotic cells, respond to ER stress by activating a set of pathways known as the unfolded protein response (UPR). The UPR is transmitted through the activation of ER resident proteins, such as protein kinase-like ER kinase (PERK), activating transcription factor 6 (ATF6) and inositol-required enzyme 1 (Irel/β). These signaling pathways induce the expression of a number of molecular chaperones such as the KDEL amino acid signature containing proteins glucose regulated protein (GRP) 78 (also Known as Bip) and 70 that play a role in assisting the correct folding of proteins in an effort to try and ameliorate the accumulation of misfolded proteins and thus alleviate ER stress (21). However, if ER stress is prolonged and the burden of misfolded proteins in the ER exceeds its folding capacity, cells are typically induced into apoptosis.

Prolonged perturbation of the ER is a powerful inducer of the C/EBP family member GADD153 (also known as CHOP) that plays a key role in the induction of programmed cell death by ER stress (10). Previous work has shown that GRP78 over-expression in Chinese hamster ovary cells attenuates both the ER stress-signal and the cell death that is observed in response to calcium ionophores (22) and blocking the expression of GRP78 by means of antisense constructs increase the lethality of agents that promote ER stress (23). Within this context, it is conceivable that cystathionine could protect against ER stress induced cell injury by increasing the induction of molecular chaperones like Grp78 or by inhibiting the induction of GADD153. In order to investigate the mechanism by which cystathionine may be protecting against ER stress induced tissue injury, an immunohistochemical analysis of liver and kidney tissues samples from the experimental groups described above was performed. PPG treatment alone did not induce expression of any KDEL signature proteins (FIG. 8, Panel A). Strong induction of KDEL, was observed, containing proteins and GADD153 by tunicamycin treatment in both liver and kidneys of the tunicamycin groups (FIG. 8, Panel B). Despite the obvious protective effects of PPG-induced hypercystathionemia, this treatment did not appear to affect the level of induction of either the KDEL containing chaperones or GADD153 indicating that cystathionine is not exerting protective effects against ER stress by modulating the UPR or by blocking the induction of GADD153.

### Example XV

### Cells Treated with Cystathionine have Increased Resistance to the Death-Promoting Effects of ER Stress

The results from the in vivo model presented above indicate that in addition to protecting against ER stress induced lipid accumulation and tissue injury, cystathionine may also play a role in promoting cell survival by attenuating the induction of apoptosis. However, examination of a direct effect of exogenously added cystathionine is not currently possible in a whole animal model because of complications with the compounds solubility and rapid kinetics of its excretion. In order to investigate the direct effects of cystathionine against ER stress-induced cell death, the cytotoxicity of tunicamycin in the human embryonic kidney cell line 293AD and the human hepatoma cell line HepG2 in the presence and absence of both PPG and exogenously added cystathionine was examined. Preliminary experiments confirmed that both of these cell lines express both CBS and CGL (data not shown). In order to investigate the possible protective effects of cystathionine in the absence of transsulfuration, these experiments were repeated with the CBS and CGL negative Chinese hamster fibroblast line a23 (6).

Cells were seeded in triplicate at ˜75% confluence and pre-treated with either PPG) (5 mM), Cystathionine (1 mM) or with just complete media (DMEM, 10% FBS) as a control. After 24 hr. pre-treatment, cells looked normal, with minimal floating cells. Fresh media with 1% FBS was then added to each well, the cells were further treated with Tm (either 5 or 10 ug)/ml. After 18 hr. treatment with Tunicamycin, cells were photographed under phase-contrast with a Zeiss Axiovert 25 microscope for evidence of dead floating cells. Cells from the group treated solely with tunicamycin became strikingly hyper-refringent, detached from the substratum and floated in the media. Conversely, No floating dead cells were observed in either the PPG or cystathionine pre-treated cells (FIG. 9, Panel B). To further evaluate a role for cystathionine in resisting the induction of cell death by tunicamycin, cell lysis was quantified by assaying lactate dehydrogenase (LDH) release. The enzyme activity was determined spectrophotometrically and the amount of enzyme leak was expressed as a percentage of the LDH activity observed in untreated control cells.

Treatment of 293AD cells with 5 μg/ml of tunicamycin resulted in significant cell death that was largely prevented by pre-treatment with PPG (FIG. 9, Panel A). Treatment of the cells with a higher dose of tunicamycin (10 μg/ml) resulted in an even more pronounced protective effect with PPG and cystathionine causing an 11 and 27-fold reduction in cell death respectively (p<0.0001). Essentially identical results were observed when this experiment was repeated using HepG2 cells (data not shown). When this experiment was repeated with the transsulfuration negative a23 cells, cystathionine was still clearly protective with a 16-fold reduction in tunicamycin induced cell death (p<0.0001) but PPG conferred no significant cytoprotection (p=0.7301). This latter observation allows us to discount the possibility that PPG itself or a metabolite thereof is responsible for the protection independent of its role in inducing elevated cystathionine. This data suggests that cystathionine promotes increased survival of cells exposed to toxic levels of ER stress.

### Example XVI

### Cystathionine Exerts its Protective Effects Against ER Stress Induced Cell Death without Modulating Expression of the UPR

To further assess the effect of cystathionine upon induction of the UPR and GADD153 by ER stress Northern and Western blotting experiments were performed. Neither PPG nor exogenously added cystathionine induced expression of the KDEL containing proteins Grp78 or Grp94 or the cell death promoting gene GADD15 in the control cells (FIG. 10, Panels A-B). Nor did either of these treatments significantly alter the scale of induction of these proteins in the presence of tunicamycin treatment. The results of these experiments are consistent with our findings in the wild type mouse model described above and further indicate that cystathionine does not exert its cytoprotective effects against ER stress induced cell death by modulating the expression of the UPR.

### Example XVII

### Cystathionine can Protect Against Thapsagargin Induced Cell Death

It was previously shown that cystathionine can protect against ER stress induced cell death induced by the glycosylation inhibitor tunicamycin. In order to investigate if this protective effect was limited to tunicamycin, it was investigated whether cystathionine (1 mM) could protect A293AD kidney cells against ER stress induced cell death mediated by the calcium metabolism inhibitor compound thapsagargin (1 uM). Cell death was monitored by measuring LDH release into culture media. In order to investigate if cystathionine was simply blocking apoptosis, the effect of thapsagargin in the presence of the known apoptosis inhibitor Z-VAD-FMK was investigated. FIG. 11 shows the pooled results of 3 independent experiments each performed in triplicate. While it is not necessary to understand the mechanism of action, it is believed that 1. No cell death was observed in the untreated or cystathionine controls; 2. Cystathionine exerted highly significant protective effects against thapsagargin induced cell death; and 3. The apoptosis inhibitor compound only reduced total cell death by approximately 20% indicating the cellular protective effects of cystathionine are not solely due to the inhibition of apoptosis.

### Example XVIII

### Cystathionine Protects Against ER Stress Induced Cell Death Independent of its Ability to Block Apoptosis

Previous work showed that cystathionine can block ER stress induced apoptosis. The acute renal necrosis seen in mice treated with tunicamycin indicates that much of the tissue damage induced by this compound is necrotic and independent of apoptosis. In order to investigate if cystathionine protects against tunicamycin (Tuc) or thapsagargin (Thap) induced cell death solely as an anti-apoptotic compound the ability of the known apoptotic inhibitor compound Z-VAD-FMK to prevent cell death induced by these treatments in HepG2 cells was investigated. FIG. 12 shows pooled results of 3 independent experiments each performed in triplicate. While it is not necessary to understand the mechanism of action, it is believed that 1. Z-VAD-FMK exerted only modest protection against cell death induced by either tunicamycin or thapsagargin (p>0.05 for both) and 2. Cystathionine exerted much greater protective effects against tunicamycin and thapsagargin indicating that much of its protective effects are independent of its role in blocking apoptosis.

### Example XVIV

### Cystathionine Protects Against Acute Tubular Necrosis Induced by the ER Stress Agent Thapsagargin

Wild type mice (WT; n=6) to a single sub-lethal intraperitoneal injection of thapsagargin in the presence and absence of the CGL inactivating compound propargylglycine (PPG). WT mice were randomly assigned to one of four treatment groups (n=6 for each group): 1). control, IP injected with PBS (See Panel A); 2). I.P. injection with PPG (See Panel B); 3). single I.P tunicamycin, with thapsagargin (See Panel C); and 4). thapsagargin+PPG (See Panel D). In groups 2 and 4, PPG treatment was maintained by daily injection until sacrifice at three days post vehicle/thapsagargin injection. After the completion of this treatment regime, all mice were anaesthetized (isoflourane/oxygen) and sacrificed by decapitation.

While it is not necessary to understand the mechanism of action, it is believed that the histological analysis revealed severe vacuolation, steatosis and acute tubular necrosis with swelling of proximal tubular epithelial cells with pyknotic nuclei and tubules with focal areas of denuded basal lamina. The histological alterations were primarily located in the proximal tubular epithelium. (See FIG. 13 showing images from mice taken from each experimental group. n=5 for each group). These morphological changes were completely ablated in the mice that were induced to accumulated cystathionine by PPG treatment.

### REFERENCES

All publications and patents mentioned in the above specification are herein incorporated by reference. Various modifications and variations of the described methods and devices of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in molecular biology/biotechnology and- or related fields are intended to be within the scope of the following claims.
1. Mudd, S. H., Levy, H. L., and Kraus, J. P. (2001) Disorders of transsulfuration. in The Metabolic and Molecular Bases of Inherited Disease (Scriver, C. R., Beaudet, A. L., Sly, W. S., Valle, D., Childs, B., Kinzler, K., and Vogelstein, B. eds.), 8 Ed., McGraw-Hill, New York. pp 2007-2056
2. Watanabe, M., Osada, J., Aratani, Y., Kluckman, K., Reddick, R., Malinow, M. R., and Maeda, N. (1995) Proc Natl Acad Sci USA 92, 1585-1589
3. Maclean K N, S. J., Kožich V, Jiang H, Greiner L S, Kraus E, Krijt J, Crnic L S, Allen R H, Stabler S P, Elleder M, Kraus J P. (2010) Molecular genetics and metabolism October-November; 101(2-3):163-71.
4. Werstuck, G. H., Lentz, S. R., Dayal, S., Hossain, G. S., Sood, S. K., Shi, Y. Y., Zhou, J., Maeda, N., Krisans, S. K., Malinow, M. R., and Austin, R. C. (2001) J Clin Invest 107, 1263-1273
5. Maclean K N, S. J., Kožich V, Jiang H, Greiner L S, Kraus E, Krijt J, Overdier K H, Collard R, Brodsky G L, Meltesen L, Crnic L S, Allen R H, Stabler S P, Elleder M, Rozen R, Patterson D, Kraus J P. (2010) Molecular genetics and metabolism October-November; 101(2-3):153-62.
6. Maclean, K. N., Kraus, E., and Kraus, J. P. (2004) J Biol Chem 279, 8558-8566
7. Rahman, S. M., Schroeder-Gloeckler, J. M., Janssen, R. C., Jiang, H., Qadri, I., Maclean, K. N., and Friedman, J. E. (2007) Hepatology 45, 1108-1117
8. Barber, T., Triguero, A., Martinez-Lopez, I., Torres, L., Garcia, C., Miralles, V. J., and Vina, J. R. (1999) J Nutr 129, 928-933
9. Finnie, J. W., and O'Shea, J. D. (1989) Pathology 21, 194-199
10. Zinszner, H., Kuroda, M., Wang, X., Batchvarova, N., Lightfoot, R. T., Remotti, H., Stevens, J. L., and Ron, D. (1998) Genes Dev 12, 982-995
11. Allen, R. H., Stabler, S. P., and Lindenbaum, J. (1993) Metabolism 42, 1448-1460
12. Bergmeyer, H. U., and Horder, M. (1980) J Clin Chem Clin Biochem 18, 521-534
13. Bligh, E. G., and Dyer, W. J. (1959) Can J Biochem Physiol 37, 911-917 14. Dickhout, J. G., Hossain, G. S., Pozza, L. M., Zhou, J., Lhotak, S., and Austin, R. C. (2005) Arterioscler Thromb Vasc Biol 25, 2623-2629
15. Wang, L., Chen, X., Tang, B., Hua, X., Klein-Szanto, A., and Kruger, W. D. (2005) Hum Mol Genet 14, 2201-2208
16. Vance, J. E., and Vance, D. E. (1985) Can J Biochem Cell Biol 63, 870-881
17. Schroder, M., and Kaufman, R. J. (2005) Annu Rev Biochem 74, 739-789
18. Koj, A., Bereta, J., Dubin, A., Kurdowska, A., Chindemi, P., and Regoeczi, E. (1986) Folia Histochem Cytobiol 24, 7-14
19. Finnie, J. W., and O'Shea, J. D. (1988) Acta Neuropathol 75, 411-421
20. Shinozuka, S., Tanase, S., and Morino, Y. (1982) European Journal of Biochemistry 124, 377-382
21. Malhotra, J. D., and Kaufman, R. J. (2007) Semin Cell Dev Biol 18, 716-731
22. Morris, J. A., Dorner, A. J., Edwards, C. A., Hendershot, L. M., and Kaufman, R. J. (1997) J Biol Chem 272, 4327-4334
23. Little, E., and Lee, A. S. (1995) J Biol Chem 270, 9526-9534
24. Hazelton, G. A., Hjelle, J. J., and Klaassen, C. D. (1986) J Pharmacol Exp Ther 237, 341-349
25. Houghton, C. B., and Cherian, M. G. (1991) J Biochem Toxicol 6, 221-228
26. Volpe, J. J., and Laster, L. (1972) Biol Neonate 20, 385-403
27. Quere, I., Paul, V., Rouillac, C., Janbon, C., London, J., Demaille, J., Kamoun, P., Dufier, J. L., Abitbol, M., and Chasse, J. F. (1999) Biochem Biophys Res Commun 254, 127-137
28. Sturman, J. A., Gaull, G., and Raiha, N. C. (1970) Science 169, 74-76
29. Gaull, G. E., Sturman, J. A., and Raiha, N. C. R. (1972) Pediatric Research 6, 538-547
30. Levonen, A. L., Lapatto, R., Saksela, M., and Raivio, K. O. (2000) Biochem J 347 Pt 1, 291-295
31. Machida, Y., Zhang, J., Hashimoto, K., Wakiguchi, H., Kurashige, T., Masuoka, N., Ubuka, T., and Kodama, H. (1995) Physiol Chem Phys Med NMR 27, 203-216
32. Zhang, J., Masuoka, N., Ubuka, T., Sugahara, K., and Kodama, H. (1995) J Inherit Metab Dis 18, 675-681
33. Pyeritz, R. E. (2000) Annu Rev Med 51, 481-510
34. Hutchinson, S., Aplin, R. T., Webb, H., Kettle, S., Timmermans, J., Boers, G. H., and Handford, P. A. (2005) J Mol Biol 346, 833-844
35. Hubmacher, D., Tiedemann, K., Bartels, R., Brinckmann, J., Vollbrandt, T., Batge, B., Notbohm, H., and Reinhardt, D. P. (2005) J Biol Chem 280, 34946-34955
36. Kuznetsov, G., Bush, K. T., Zhang, P. L., and Nigam, S. K. (1996) Proc Natl Acad Sci USA 93, 8584-8589
37. Ryu, E. J., Harding, H. P., Angelastro, J. M., Vitolo, 0. V., Ron, D., and Greene, L. A. (2002) J Neurosci 22, 10690-10698
38. Aw, T. Y., Ookhtens, M., and Kaplowitz, N. (1984) J Biol Chem 259, 9355-9358
39. Ghibelli, L., Fanelli, C., Rotilio, G., Lafavia, E., Coppola, S., Colussi, C., Civitareale, P., and Ciriolo, M. R. (1998) Faseb J 12, 479-486.
40. Ghibelli, L., Coppola, S., Rotilio, G., Lafavia, E., Maresca, V., and Ciriolo, M. R. (1995) Biochem Biophys Res Commun 216, 313-320
41. Gurney, J. G., Davis, S., Severson, R. K., Fang, J. Y., Ross, J. A., and Robison, L. L. (1996) Cancer 78, 532-541
42. Gurney, J. G., Ross, J. A., Wall, D. A., Bleyer, W. A., Severson, R. K., and Robison, L. L. (1997) J Pediatr Hematol Oncol 19, 428-432
43. Gjessing, L. R. (1963) Scand J Clin Lab Invest 15, 601-602
44. Geiser, C. F., and Efron, M. L. (1968) Cancer 22, 856-860
45. Helson, L., Fleisher, M., Bethune, V., Murphy, M. L., and Schwartz, M. K. (1972) Clin Chem 18, 613-615
46. Klein, C. E., Roberts, B., Holcenberg, J., and Glode, L. M. (1988) Cancer 62, 291-298

What is claimed:

1-23. (canceled)

24. A method for treating a kidney condition in a subject in need thereof comprising administering a pharmaceutically acceptable composition comprising at least one of a compound comprising cystathionine or a derivative or metabolite thereof; and a cystathionine gamma-lyase (γCGL) inhibitor; and treating the kidney condition.

25. The method according to claim 24, wherein the cystathionine derivative or metabolite thereof comprises perhydro-1,4-thiazepine-3,5-dicarboxylic acid, a cystathionine mono-oxo acid, a cystathionine ketimine, a cystathionine sulfoxide, and an N-acetylcystathionine sulfoxide.

26. The method according to claim 24, wherein the γCGL inhibitor comprises propargylglycine (PPG).

27. The method according to claim 24, wherein the γCGL inhibitor comprises an antibody or monoclonal antibody capable of reducing expression or activity of γCGL.

28. The method according to claim 24, wherein the γCGL inhibitor reduces expression or activity of γCGL, elevating cystathionine levels in the subject to treat the kidney condition.

29. The method according to claim 24, wherein the pharmaceutically acceptable composition comprises cystathionine.

30. The method according to claim 24, wherein the kidney condition is caused by one or more of kidney transplantation (renal/cyclosporine), adverse effects of other organ transplantations, a toxin-induced condition, drug toxicity, a blood transfusion, ischemia reperfusion-related kidney injury, a side-effect of trauma, muscle injury side effects, major surgery, septic shock, severe low blood pressure for thirty minutes or greater, antibiotic-related side effects or toxicity, antifungal agent side effects, diagnostic dye side effects, diabetes or infection in the subject.

31. The method according to claim 30, wherein the infection in the subject comprises a viral or bacterial infection.

32. The method according to claim 30, wherein the kidney condition comprises acute tubular necrosis.

33. The method according to claim 30, wherein the kidney condition comprises acute kidney failure.

34. The method according to claim 24, wherein the pharmaceutically acceptable composition is administered parenterally, by inhalation, intraperitoneally, intramuscularly, subcutaneously or orally.

35. The method according to claim 24, wherein administration comprises daily, every other day, bi-weekly, weekly, bi-monthly or monthly administration to the subject.

36. The method according to claim 24, further comprising an anti-apoptotic agent, anti-microbial agent, or anti-rejection agent.

37. The method according to claim 24, further comprising one or more anti-rejection agent.

38. The method according to claim 24, wherein administration of the pharmaceutically acceptable composition reduces apoptotic cell death in the kidney.

39. A method for maintaining calcium homeostasis in a subject in need thereof comprising administering a pharmaceutically acceptable composition comprising at least one of a compound comprising cystathionine or a derivative or metabolite thereof; and a cystathionine γ-lyase (γCGL) inhibitor; and maintaining calcium homeostasis in the subject.

40. The method according to claim 39, wherein the subject comprises a subject having acute kidney failure.

41. The method according to claim 39, wherein the pharmaceutically acceptable composition comprises cystathionine and the cystathionine level in the treated subject is elevated compared to a control subject not receiving the pharmaceutically acceptable composition.

42. The method according to claim 39, wherein the pharmaceutically acceptable composition comprises a γCGL inhibitor and the cystathionine level in the treated subject is elevated in the subject compared to a control subject not receiving the pharmaceutically acceptable composition.

43. A kit comprising a pharmaceutically acceptable composition comprising a γCGL inhibitor and at least one container.
